(19) United States
(12) Patent Application Publication
(10) Pub. No.: US 2016/0371950 A1
(43) Pub. Date: Dec. 22, 2016

(54) INFORMATION PROCESSING APPARATUS, INFORMATION PROCESSING METHOD, AND PROGRAM

(71) Applicant: NORITSU PRECISION CO., LTD., Wakayama-shi, Wakayama (JP)

(72) Inventor: Toru Yasukawa, Wakayama-shi (JP)

(73) Assignee: NORITSU PRECISION CO., LTD., Wakayama-shi, Wakayama (JP)

(21) Appl. No.: 15/122,230

(22) PCT Filed: Jan. 22, 2015
(86) PCT No.: PCT/JP2015/051634

§ 371 Date: Aug. 29, 2016

(30) Foreign Application Priority Data
Mar. 6, 2014 (JP) 2014-044053

(51) Int. Cl. G08B 21/04; G06K 9/00; H04N 7/18; A61B 5/11; A61B 5/00

(52) U.S. Cl. CPC G08B 21/0476; A61B 5/1115; A61B 5/746; A61B 5/0077; H04N 7/183; G06K 9/00771; G08B 21/043

(57) ABSTRACT

A technology is provided, which reduces a misreport of a watching system due to a non-target person's image getting contained in a captured image. An information processing apparatus includes an image acquiring unit to acquire a captured image of an imaging apparatus for watching a watching target person, a behavior detection unit to detect a behavior of the watching target person, a non-target person detection unit to detect an existence of a non-target person other than the watching target person, and a report unit to execute reporting to notify that the behavior of the watching target person is detected upon detecting the behavior of the watching target person while not detecting an existence of the non-target person, and to quit reporting to notify that the behavior of the watching target person is detected while detecting the existence of the non-target person.

### TECHNICAL FIELD

The present invention pertains to an information processing apparatus, and information processing method, and a program.

### BACKGROUND ART

For example, Patent document 1 proposes a technology for determining a get-up behavior on abed of a patient by utilizing a captured image. In a get-up monitoring apparatus proposed in Patent document 1, a watching area for determining the get-up behavior of a sleeping patient lying in the bed is set immediately above the bed. The get-up monitoring apparatus is configured such that a camera captures an image of the watching area from sideways of the bed, and the get-up behavior of the patient is detected when a size of a patient's image area occupying the watching area in the captured image obtained from the camera is smaller than an initial value.

### CITATION LIST

### Patent Literature

[Patent document 1] Patent Literature 1: JP 2011-005171

### SUMMARY OF INVENTION

### Technical Problem

In recent years, there has been an annually increasing tendency of accidents that watching target persons instanced by inpatients, assisted-living residents and care receivers fall down or come down from beds and of accidents caused by wandering of dementia patients. A watching system, which is developed as a method of preventing those accidents, is configured to detect watching target person's behaviors instanced by a get-up state, a sitting-on-bed-edge state and a leaving-bed state by capturing an image of the watching target person with an imaging apparatus (camera) installed indoors and analyzing the captured image as exemplified in Patent document 1.

The watching system described above, when a non-target person other than the watching target person enters an image capture range of an imaging apparatus, has a possibility of mistaking a behavior of the non-target person for a behavior of the watching target person and being disabled from adequately detecting the behaviors of the watching target person. If the behavior of the non-target person is erroneously detected as the behavior of the watching target person, the watching target person does not actually behave such as getting up, and nevertheless the watching system results in reporting the behavior of the watching target person, based on the mis-detection. Proposed as a countermeasure for preventing such a misreport is a method for preventing the behavior of the watching target person from being erroneously detected by distinguishing between the behavior of the watching target person and the behavior of the non-target person as hitherto exemplified by Patent document 1. There is, however, a limit to distinction between the behavior of the watching target person and the behavior of the non-target person, and the watching system has a difficulty of preventing the misreport due to the non-target person's image getting contained in the captured image.

According to one aspect, the present invention is devised in view of these points and aims at providing a technology for preventing a misreport of a watching system due to a captured image containing an image of a non-target person other than a watching target person.

### Solution to Problem

The present invention adopts following configurations in order to solve the problems described above.

To be specific, an information processing apparatus according to one aspect of the present invention includes an image acquiring unit to acquire a captured image captured by an imaging apparatus installed for watching a watching target person; a behavior detection unit to detect a behavior of the watching target person by analyzing the acquired captured image; a non-target person detection unit to detect an existence of a non-target person other than the watching target person in a range where the behaviors of the watching target person can be watched; and a report unit to execute reporting to notify that the behavior of the watching target person is detected upon detecting the behavior of the watching target person while not detecting an existence of the non-target person, and to quit reporting to notify that the behavior of the watching target person is detected while detecting the existence of the non-target person.

The information processing apparatus according to the configuration described above detects the behavior of the watching target person by analyzing the captured image captured by the imaging apparatus installed for watching the behaviors of the watching target person. The information processing apparatus executes the report to notify that the behavior of the watching target person is detected upon detecting the behavior of the watching target person. Namely, the information processing apparatus according to the configuration described above builds up a watching system for watching the watching target person on the basis of the image analysis.

The inventors of the present invention found out the following. Specifically, a scene with no person to watch the watching target person is exactly the scene that requires watching by the watching system. In contrast with this, in a scene where the image of the non-target person other than the watching target person gets contained in the captured image, the watching target person is watched by the non-target person, and there is a high possibility of not requiring the watching by the watching system. Namely, the inventors of the present invention found out that the report of the watching system may not be executed on condition that the non-target person exists in a range where the behaviors of the watching target person can be watched.

Such being the case, the information processing apparatus according to the configuration described above detects the existence of the non-target person other than the watching target person in the range where the behaviors of the watching target person can be watched. While detecting the existence of the non-target person, the information processing apparatus according to the configuration described above quits executing the report to notify that the behavior of the watching target person is detected. It thereby follows that the report of the watching system is not executed in the scene where the image of the non-target person other than the watching target person gets contained in the captured image. It is therefore feasible to prevent the misreport of the watching system, the misreport being derived from the non-target person's image getting contained in the captured image.

Note that the “watching target person” is a target person whose behaviors are watched and is exemplified by an inpatient, an assisted-living resident and a care receiver. The non-target persons each becoming a trigger to quit the report of the watching system may be any persons if other than the watching target person and encompass, e.g., persons who watch the watching target person. The persons who watch the watching target person are exemplified by nurses, care facility staff and caregivers. The behaviors of the watching target person becoming the target to be detected by the information processing apparatus may be any behaviors instanced by a get-up state on the bed, a sitting-on-bed-edge state, an over-bed-fence state, a come-down state from the bed and a leaving-bed state. Herein, the sitting-on-bed-edge state indicates such a state that the watching target person sits on an edge of the bed. The over-bed-fence state represents a state where the watching target person leans over a fence of the bed.

As another mode of the information processing apparatus according to one aspect, the information processing apparatus may further include a history generation unit to generate a history of the behaviors of the watching target person, the behaviors being detected by the behavior detection unit. This configuration enables archiving of the history of the behaviors detected by the watching system.

As still another mode of the information processing apparatus according to one aspect, the behavior detection unit may detect the behaviors of the watching target person irrespective of whether the existence of the non-target person is detected or not, and the history generation unit may generate the history of the behaviors of the watching target person irrespective of whether the existence of the non-target person is detected or not, the behaviors being detected by the behavior detection unit. This configuration enables archiving of the history of the behaviors detected by the watching system without depending on whether to quit executing the report about the detection of the behavior of the watching target person.

As yet still another mode of the information processing apparatus according to one aspect, the non-target person detection unit may detect the existence of the non-target person by analyzing the acquired captured image. In a scene where the image of the non-target person gets contained in the captured image, as described above, there is a high possibility of not requiring the watching by the watching system. On the other hand, the watching system erroneously detects the behavior of the watching target person because of the non-target person's image getting contained in the captured image, and has a high possibility of resultantly reporting that the behavior of the watching target person is detected. This configuration can prevent the misreport about the detection of the behavior of the watching target person by quitting the execution of the report by the watching system in such a scene.

As yet another mode of the information processing apparatus according to one aspect, the imaging apparatus may capture an image of an object serving as a reference for the behaviors of the watching target person together with the image of the watching target person, and the image acquiring unit may acquire the captured image containing depth information indicating a depth of each of pixels within the captured image. The behavior detection unit may detect the watching target person's behavior pertaining to the object by determining whether a positional relation in a real space between the watching target person and the object satisfies a predetermined condition or not, based on the depth of each of the pixels within the captured image, the depth being indicated by the depth information.

The depth of each of the pixels indicates a depth of an object contained in each of the pixels. Therefore, according to this configuration, the behavior of the watching target person can be detected by taking account of a state in the real space. Note that the object serving as a reference for the behaviors of the watching target person may be properly selected corresponding to the scene of watching the watching target person. For example, the reference object for the behaviors is set to the bed in the case of watching the get-up state and other equivalent states of the watching target person on the bed.

As yet another mode of the information processing apparatus according to one aspect, the information processing apparatus may further include a foreground extraction unit to extract a foreground area of the captured image from a difference between the captured image and a background image set as a background of the captured image. The behavior detection unit may detect the watching target person's behavior pertaining to the object by determining whether the positional relation in the real space between the watching target person and the object satisfies the predetermined condition or not while utilizing, as a position of the watching target person, a position of the object in the real space with its image being contained in the foreground area, the latter position being specified based on the depth of each of the pixels in the foreground area.

According to this configuration, the foreground area of the captured image can be specified by extracting the difference between the background image and the captured image. This foreground area is an area with occurrence of a variation from the background image. Consequently, the foreground area includes an area with occurrence of a variation caused by a motion of the watching target person, in other words, an area covering an existence of a region in motion (which will hereinafter be termed a “motion region”) with respect to body regions of the watching target person as an image pertaining to the watching target person. Hence, the position of the motion region of the watching target person in the real space can be specified by referring to the depth of each of the pixels within the foreground area indicated by the depth information.

Such being the case, the information processing apparatus according to the configuration described above utilizes, as the position of the watching target person, the position, specified based on the depth of each of the pixels within the foreground area, of the object with its image getting contained in the foreground area in the real space, thereby determining whether a positional relation in the real space between the watching target person and the object satisfies a predetermined condition. More specifically, the predetermined condition for detecting the behavior of the watching target person is set on the assumption that the foreground area pertains to the behaviors of the watching target person. The information processing apparatus according to the configuration described above is thereby enabled to detect the behaviors pertaining to the object for the watching target person.

Herein, the process of extracting the foreground area is a mere calculation of a difference between the background image and the captured image. Therefore, according to the configuration described above, the behaviors of the watching target person can be detected by a simple method corresponding to states in the real space.

As a further mode of the information processing apparatus according to one aspect, the information processing apparatus may be connected to a receiver to receive information transmitted from a wireless communication device carried by the non-target person. The receiver may be disposed in a communication-enabled place with the wireless communication device carried by the non-target person existing in the range where the watching target person can be watched. The non-target person detection unit may detect the existence of the non-target person upon an event that the receiver receives the information transmitted from the wireless communication device. According to this configuration, the information transmitted from the wireless communication device is received, whereby the existence of the non-target person can be detected. The non-target person can be therefore detected not depending on sophisticated information processing such as image recognition.

As a still further mode of the information processing apparatus according to one aspect, the non-target person detection unit may determine whether the non-target person is a predetermined person or not by referring to the information transmitted from the wireless communication device. The report unit may quit the report to notify that the behavior of the watching target person is detected when the non-target person is the predetermined person while detecting the existence of the non-target person.

Some of the non-target persons entering a watching-enabled range for the watching target person do not pay attention to the watching target person as the case may be. It is not therefore necessarily preferable to quit the report of the watching system with respect to every non-target person. According to the configuration described above, it is feasible to quit executing the report of the watching system only when the non-target person entering in the watching-enabled range for the watching target person is the predetermined person. In other words, it is feasible to prevent the execution of the report from being quitted when the non-target person deemed unpreferable to quit the report of the watching target person enters the watching-enabled range.

As a yet further mode of the information processing apparatus according to one aspect, the information processing apparatus may be connected to a nurse call system for calling a person who watches the watching target person. The report unit may perform calling via the nurse call system as the report to notify that the behavior of the watching target person is detected. According to this configuration, it is possible to report the detection of the behavior of the watching target person by the watching system via the nurse call system.

It is to be noted that an additional mode of the information processing apparatus according to the respective modes described above may be an information processing system realizing the respective configurations described above, may also be an information processing method, may further be a program, and may yet further be a non-transitory storage medium recording a program, which can be read by a computer, other apparatuses and machines. Herein, the storage medium readable by the computer etc is a medium that accumulates the information such as the program electrically, magnetically, mechanically or by chemical action. Moreover, the information processing system may be realized by one or a plurality of information processing apparatuses.

For example, an information processing method according to one aspect of the present invention is an information processing method to be executed by a computer according to one aspect of the present invention, the method comprising a step of acquiring a captured image captured by an imaging apparatus installed for watching a watching target person; a step of detecting a behavior of the watching target person by analyzing the acquired captured image; a step of detecting an existence of a non-target person other than the watching target person in a range where the behaviors of the watching target person can be watched; a step of executing a report to notify that the behavior of the watching target person is detected upon detecting the behavior of the watching target person while not detecting an existence of the non-target person; and a step of quitting the report to notify that the behavior of the watching target person is detected while detecting the existence of the non-target person.

For instance, a program according to one aspect of the present invention is a program to cause a computer to execute a step of acquiring a captured image captured by an imaging apparatus installed for watching a watching target person; a step of detecting a behavior of the watching target person by analyzing the acquired captured image; a step of detecting an existence of a non-target person other than the watching target person in a range where the behaviors of the watching target person can be watched; a step of executing a report to notify that the behavior of the watching target person is detected upon detecting the behavior of the watching target person while not detecting an existence of the non-target person; and a step of quitting the report to notify that the behavior of the watching target person is detected while detecting the existence of the non-target person.

### Advantageous Effects of Invention

According to the present invention, it is feasible to reduce the misreport of the watching system due to the captured image containing the image of the non-target person other than the watching target person.

### BRIEF DESCRIPTION OF DRAWINGS

FIG. 1 is a view illustrating one example of a scene to which the present invention is applied;

FIG. 2 is a diagram illustrating a hardware configuration of an information processing apparatus according to an embodiment;

FIG. 3 is a view depicting depths acquired by a camera according to the embodiment;

FIG. 4 is a view illustrating a relationship between the depths acquired by the camera according to the embodiment and an object;

FIG. 5 is a view illustrating one example of a captured image in which gray values of the respective pixels are specified corresponding to the depths of the pixels;

FIG. 6 is a diagram illustrating a functional configuration of the information processing apparatus according to the embodiment;

FIG. 7 is a diagram illustrating state transitions of a report mode of the information processing apparatus according to the embodiment;

FIG. 8 is a flowchart illustrating a processing procedure pertaining to the state transitions of the information processing apparatus according to the embodiment;

FIG. 9 is a view schematically illustrating the captured image acquired by the camera according to the embodiment;

FIG. 10 is a flowchart illustrating a processing procedure pertaining to watching by the information processing apparatus according to the embodiment;

FIG. 11 is a view illustrating a screen to be displayed when the information processing apparatus according to the embodiment watches the watching target person;

FIG. 12 is a view illustrating a three-dimensional distribution of objects within an image capture range, which are specified based on the depth information contained in the captured image;

FIG. 13 is a view illustrating the three-dimensional distribution of a foreground area extracted from the captured image;

FIG. 14 is a view schematically illustrating a detection area for the watching system according to the embodiment to detect a get-up state;

FIG. 15 is a view schematically illustrating a detection area for the watching system according to the embodiment to detect a leaving-bed state;

FIG. 16 is a view schematically illustrating a detection area for the watching system according to the embodiment to detect a sitting-on-bed-edge state;

FIG. 17 is a diagram illustrating history information according to the embodiment;

FIG. 18 is a diagram illustrating a relationship between a degree of spread of the area and dispersion;

FIG. 19A is a view illustrating a method of detecting a non-target person in a modified example;

FIG. 19B is a view illustrating the method of detecting the non-target person in the modified example; and

FIG. 20 is a diagram illustrating a hardware configuration of the watching system according to the modified example.

### DESCRIPTION OF EMBODIMENTS

An embodiment (which will hereinafter be also termed “the present embodiment”) according to one aspect of the present invention will hereinafter be described based on the drawings. However, the present embodiment, which will hereinafter be explained, is no more than an exemplification of the present invention in every point. As a matter of course, the invention can be improved and modified in a variety of forms without deviating from the scope of the present invention. Namely, on the occasion of carrying out the present invention, a specific configuration corresponding to the embodiment may properly be adopted.

Note that data occurring in the present embodiment are, though described in a natural language, specified more concretely by use of a quasi-language, commands, parameters, a machine language and other equivalent languages, which are recognizable to a computer.

### §1 Example of Applied Situation

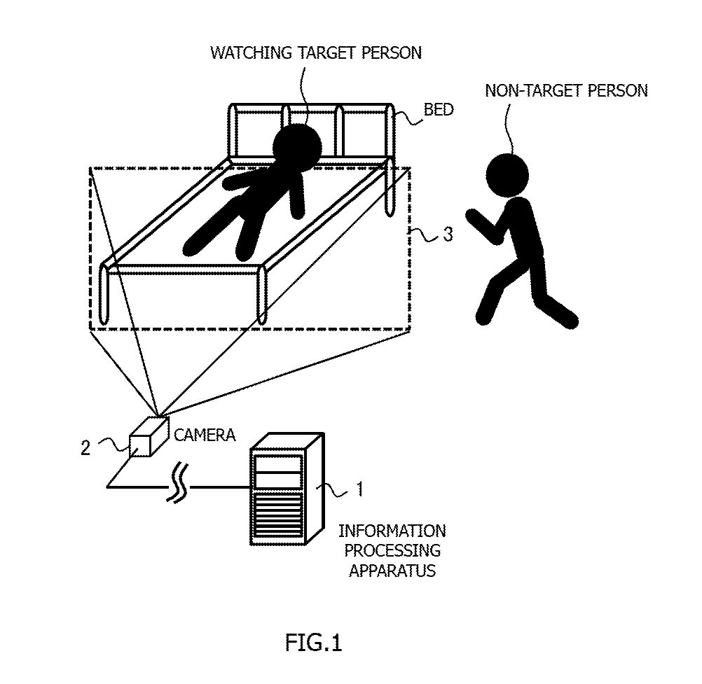

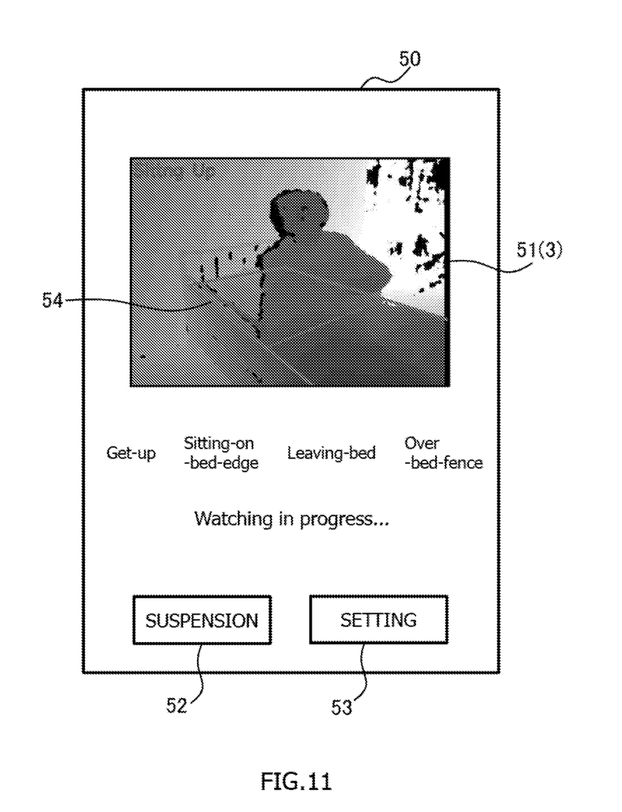

At first, a situation to which the present invention is applied will be described by using FIG. 11. FIG. 1 schematically illustrates one example of the situation to which the present invention is applied. The present embodiment assumes a situation of watching a behavior of an inpatient in a medical treatment facility a nursing facility or a tenant in a nursing facility as a watching target person. A person (who will hereinafter be also referred to as a “user”) who watches a watching target person is exemplified by a nurse or a care facility staff member. The user watches behavior of the watching target person on a bed by utilizing a watching system including an information processing apparatus 1 and a camera 2. However, the watching target person, the user and the watching situation may be properly selected corresponding to the embodiment.

The camera 2 of the watching system according to the embodiment captures of an image of the behavior of the watching target person. The camera 2 corresponds to an “imaging apparatus” according to the present invention. In the embodiment, the camera 2 is installed for watching the behavior of the watching target person on the bed. Note that a placeable position of the camera 2 but may not be limited to a specific position but may be properly selected corresponding to the embodiment. In the embodiment, the camera 2 is installed in front of the bed in a longitudinal direction.

The information processing apparatus 1 according to the embodiment obtains a captured image 3 that is captured by this camera 2. The information processing apparatus 1 detects the behavior of the watching target person by analyzing the obtained captured image 3. The information processing apparatus 1, corresponding to the detection of the behavior of the watching target person, executes a report to notify the user that the behavior of the watching target person has been detected. According to the embodiment, the watching system is thus enabled to watch the watching target person.

In the embodiment, however, the behavior of the watching target person is detected by analyzing the captured image 3, and consequently, as illustrated in FIG. 1, when a non-target person other than the watching target person enters an image capture range, the information processing apparatus 1 might have a possibility of detecting behavior of the non-target person erroneously as the behavior of the watching target person. If done so, it follows that the information processing apparatus 1 executes the report to notify the user of the detection of the behavior, based on an erroneous detection result. Accordingly, the watching target person does not take any behavior, and nevertheless the information processing apparatus 1 has the possibility of executing the report to notify the user of the detection of the behavior due to the non-target person's image being contained in the captured image 3.

The information processing apparatus 1 according to the embodiment determines, for preventing the misreport described above, whether the non-target person other than the watching target person exists in a range where the behavior of the watching target person can be watched. While the existence of the non-target person remains undetected, the information processing apparatus 1 executes the report to notify the user that the behavior has been detected, corresponding to the detection of the behavior of the watching target person. On the other hand, while the existence of the non-target person remains detected, the information processing apparatus 1 quits executing the report to notify the user of the detection of the behavior even when having detected the behavior of the watching target person. In the embodiment, it is thereby possible to quit executing the report to notify the user of the behavior in a situation where the image of the watching target person is contained in the captured image 3, and the watching system can be therefore prevented from executing the misreport due to the non-target person's image being contained in the captured image 3.

Note that the non-target person other than the watching target person may be any person if other than the watching target person, and is exemplified by the nurse, the care facility staff member and a caregiver who watch the watching target person. The non-target person may further include hospital visitors for the watching target person, workers conducting works in a watching environment, and other equivalent persons.

### §2 Example of Configuration

### Hardware Configuration

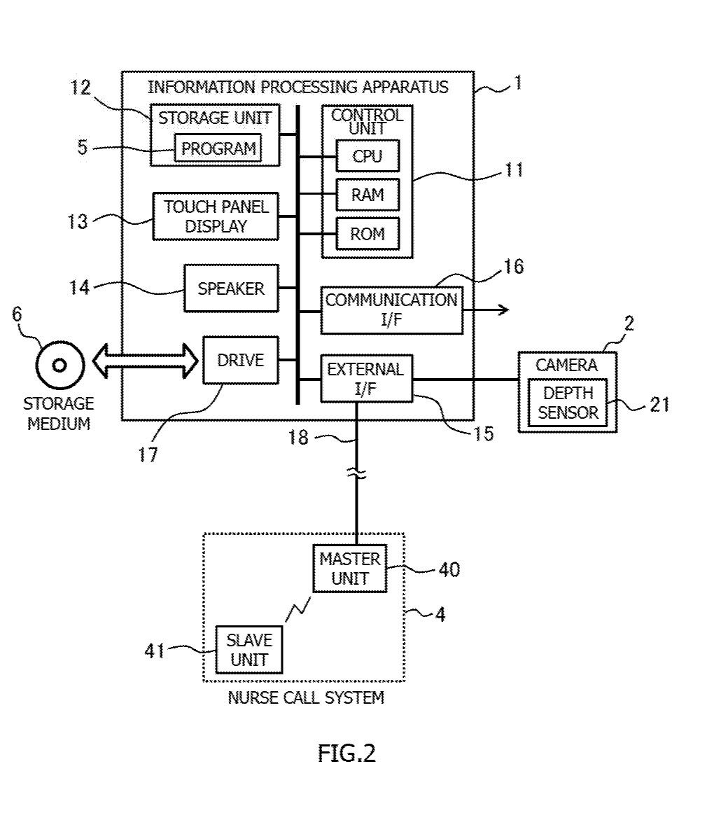

Next, a hardware configuration of the information processing apparatus 1 will hereinafter be described by using FIG. 2. FIG. 2 illustrates the hardware configuration of the information processing apparatus 1 according to the present embodiment. The information processing apparatus 1 is, as illustrated in FIG. 2, a computer including: a control unit 11 containing a CPU, a RAM (Random Access Memory) and a ROM (Read Only Memory); a storage unit 12 storing a program 5 and other equivalent programs executed by the control unit 11; a touch panel display 13 for displaying and inputting the images; a speaker 14 for outputting sounds; an external interface 15 for establishing a connection with an external device; a communication interface 16 for performing communications via a network; and a drive 17 for reading the program stored on a storage medium 6, which are all electrically interconnected. In FIG. 2 the communication interface and the external interface are abbreviated to the “communication I/F” and the “external I/F” respectively.

Note that as for the specific hardware configuration of the information processing apparatus 1, the components thereof can be properly omitted, replaced and added corresponding to the embodiment. For instance, the control unit 11 may include a plurality of processors. The touch panel display 13 may be replaced by, e.g., an input device and a display device, which are separately independently connected. For example, the speaker 14 may be omitted. The speaker 14 may also be connected to the information processing apparatus 1, e.g., not as an internal device but as an external device of the information processing apparatus 1. The camera 2 may also be built in the information processing apparatus 1.

The information processing apparatus 1 includes a plurality of external interfaces 15 and may be thus connected to a plurality of external devices. In the embodiment, the information processing apparatus 1 is connected to the camera 2 via the external I/F 15. The camera 2 according to the embodiment is installed for watching the behavior of the watching target person on the bed. The camera 2 includes a depth sensor 21 for measuring a depth of an object. A type and a measuring method of the depth sensor 21 may be adequately selected corresponding to the embodiment. For example, the depth sensor 21 can be instanced by a sensor of a TOF (Time Of Flight) method and other equivalent method.

Note that a place (e.g., a patient' room in the medical treatment facility) in which to watch the watching target person is a place equipped with the bed of the watching target person, in other words, a place where the watching target person goes to bed. this therefore leads to a possibility that the watching target person is watched in a dark place. Such being the case, it is preferable to use, as the depth sensor 21, an infrared-ray depth sensor for measuring a depth based on irradiation of infrared-rays in order to acquire the depth without being affected by brightness in an image capture location. A comparatively inexpensive imaging apparatus including this type of infrared-ray depth sensor can be exemplified by Kinect of Microsoft Corp., Xtion of ASUSTeK Computer Inc., and CARMINE of PrimeSense Inc.

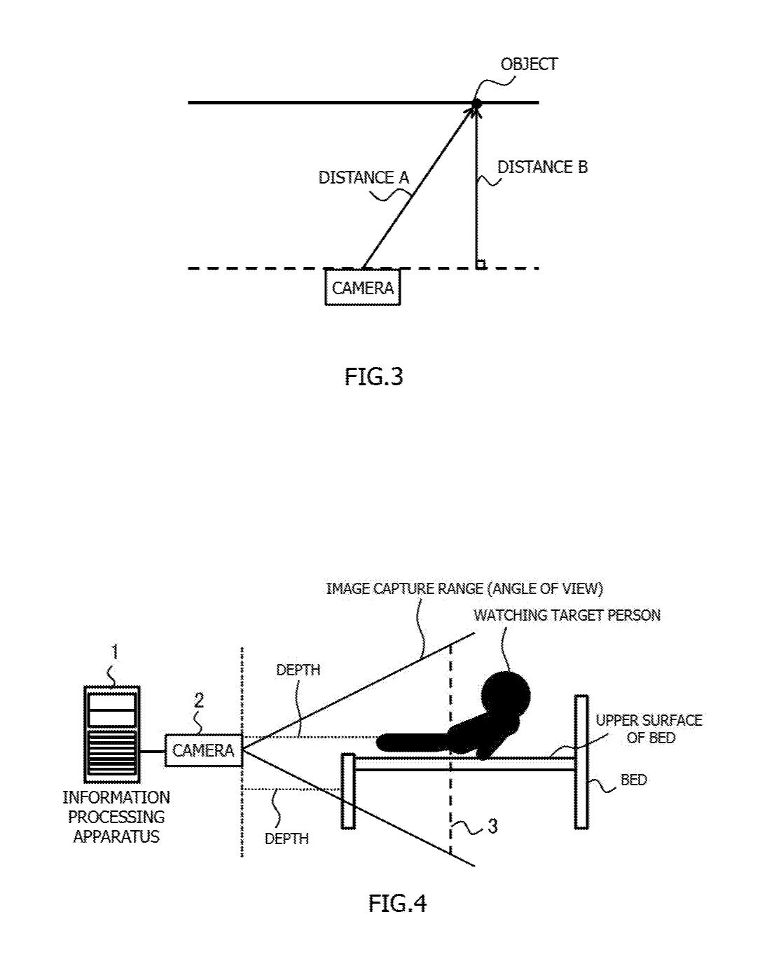

Herein, an in-depth description of the depth measured by the depth sensor 21 according to the embodiment will hereinafter be made by using FIGS. 3-5. FIG. 3 depicts one example of a distance that can be dealt with as the depth according to the embodiment. The “depth” represents a depth of the object. As depicted in FIG. 3, the depth of the object may be expressed by a distance A of a straight line between, the camera and the object, and may also be expressed by a distance B of a vertical line drawn from a horizontal axis with respect to the object of the camera.

Namely, the depth according to the embodiment may be equivalent to the distance A and may also be equivalent to the distance B. The present embodiment is to deal with the distance B as the depth. The distance A and the distance B are, however, interchangeable by utilizing, e.g., the Pythagoras theorem and other equivalent theorems. A description using the distance B given from now on can be applied directly to the distance A. The use of these depths enables, as illustrated in FIG. 4, the behavior of the watching target person to be detected by taking account of a state in a real space.

FIG. 4 illustrates a relationship in the real space between the depths acquired by the camera 2 according to the second embodiment and the object. FIG. 4 depicts a scene with the camera 2 being viewed from the side. An up-and-down direction in FIG. 4 therefore corresponds to a heightwise direction of the bed. A left-and-right direction in FIG. 4 corresponds to a longitudinal direction of the bed, and a direction vertical to a sheet surface of FIG. 4 corresponds to a widthwise direction of the bed.

As illustrated in FIG. 4, the camera 2 can acquire the depths corresponding to respective pixels within the captured image 3 by using the depth sensor 21. The captured image 3 acquired by the camera 2 therefore contains depth information representing the depth obtained per pixel. Note that a data format of the captured image 3 containing the depth information may not be limited to a specific format but may be properly selected corresponding to the embodiment. The captured image 3 may be, e.g., data indicating the depths of the objects within the image capture range and may also be such data (e.g., a depth map) that the depths of the objects within the image capture range are distributed two-dimensionally. The captured image 3 may contain RGB images together with the depth information. Further, the captured image 3 may be a moving image and may also be a static image.

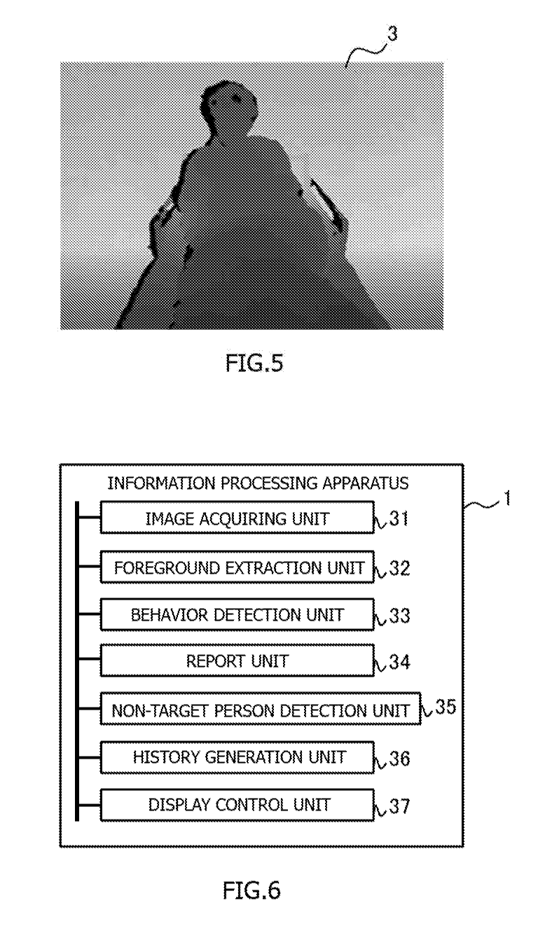

FIG. 5 illustrates one example of the captured image 3 described above. The captured image 3 depicted in FIG. 5 is an image formed such that gray values of the respective pixels are determined corresponding to the depths of the pixels. More blackish pixels indicate being closer to the camera 2. While on the other hand, more whitish pixels indicate being remoter from the camera 2. According to the depth information, it is feasible to specify a position of the object within the image capture range in the real space (three-dimensional space). The information processing apparatus 1 is thereby enabled to detect the behavior of the watching target person by utilizing the depth information while taking account of the state of the real space.

The information processing apparatus 1 is, as illustrated in FIG. 2, connected to a nurse call system 4 via the external interface 15. A hardware configuration and a functional configuration of the nurse call system 4 may be adequately selected corresponding to the embodiment. The nurse call system 4 is an apparatus for calling the user (the nurse, the care facility staff member and other equivalent persons) of the watching system that watches the watching target person, and may also be an apparatus known as the nurse call system. The nurse call system 4 according to the embodiment includes a master unit 40 connected via a wire 18 to the information processing apparatus 1, and a slave unit 41 capable of performing wireless communication with this master unit 40.

The master unit 40 is installed in, e.g., a station for the users. The master unit 40 is used mainly for calling the user existing in the station. On the other hand, the slave unit 41 is generally carried around by the user. The slave unit 41 is used for calling the user who carries the slave unit 41 around. The master unit 40 and the slave unit 41 may be equipped with speakers for outputting sounds to notify each other about various matters. Both of the master unit 40 and the slave unit 41 may also be equipped with microphones to enable verbal exchanges with the watching target person via the information processing apparatus 1 and other equivalent apparatuses. The information processing apparatus 1 connects to these units installed in the facility for the nurse call system 4 and other equivalent systems via the external interface 15, and may thus execute a variety of reports by cooperating with the units.

Note that the program 5 is a program causing the information processing apparatus 1 to execute processes contained in an operation that will be described later on, and corresponds to a “program” according to the present invention. The program 5 may be recorded on a storage medium 6. The storage medium 6 is a non-transitory storage medium that accumulates information instanced by the program electrically, magnetically, optically, mechanically or by chemical action so that the computer, other apparatuses and machines can read the information instanced by the recorded program. The storage medium 6 corresponds to a “non-transitory storage medium” according to the present invention. Note that FIG. 2 illustrates a disk type storage medium instanced by a CD (Compact Disk) and a DVD (Digital Versatile Disk) by way of one example of the storage medium 6. It does not, however, mean that the type of the storage medium 6 is limited to the disk type, and other types excluding the disk type may be available. The storage medium other than the disk type can be exemplified by a semiconductor memory instanced by a flash memory.

The information processing apparatus 1 may involve using, in addition to, e.g., an apparatus designed for an exclusive use for a service to be provided, general-purpose apparatuses instanced by a PC (Personal Computer) and a tablet terminal. The information processing apparatus 1 may be implemented by one or a plurality of computers.

### Example of Functional Configuration

Next, a functional configuration of the information processing apparatus 1 will be described by using FIG. 6. FIG. 6 illustrates the functional configuration of the information processing apparatus 1 according to the embodiment. The control unit 11 provided in the information processing apparatus 1 according to the embodiment deploys the program 5 stored in a storage unit 12 onto the RAM. The control unit 11 interprets and executes the program 5 deployed onto the RAM, thereby controlling the respective components. Through this operation, the information processing apparatus 1 according to the embodiment functions as the computer including an image acquiring unit 31, a foreground extraction unit 32, a behavior detection unit 33, a report unit 34, a non-target person detection unit 35, a history generation unit 36, and a display control unit 37.

The image acquiring unit 31 acquires the captured image 3 captured by the camera 2. The behavior detection unit 33 detects the behavior of the watching target person by analyzing the captured image 3 to be acquired. In the embodiment, the behavior detection unit 33 detects, as will be mentioned later on, a get-up state on the bed, a leaving-bed state, a sitting-on-bed-edge state, and an over-bed-fence state.

The non-target person detection unit 35 detects existence of the non-target person other than the watching target person in the range where the behavior of the watching target person can be watched. The report unit 34, when the non-target person detection unit 35 does not yet detect the existence of the non-target person, executes the report to notify the user of the detection of the behavior, corresponding to an event that the behavior detection unit 33 has detected the behavior of the watching target person. Whereas when the non-target person detection unit 35 detects the existence of the non-target person, the report unit 34 quits executing the report to notify the user of the detection of the behavior. In other words, in this instance, the report unit 34 does not execute the report to notify the user of the detection of the behavior even when the behavior detection unit 33 detects the behavior of the watching target person.

Note that the foreground extraction unit 32 extracts a foreground area of the captured image 3 from a difference between a background image set as a background of the captured image 3 and the captured image 3. The behavior detection unit 33 may detect the behavior of the watching target person by making use of this foreground area. The history generation unit 36 generates a history of the behaviors of the watching target person, which have been detected by the behavior detection unit 33. The display control unit 37 controls screen display of the touch panel display 13.

Respective functions will be described in detail in “Operational Example” that will be made later on. Herein, the embodiment discusses an instance in which each of these functions is attained by the general-purpose CPU. Some or the whole of these functions may, however, be attained by one or a plurality of dedicated processors. As for the functional configuration of the information processing apparatus 1, the functions may be properly omitted, replaced and added corresponding to the embodiment. For example, when not displaying the screen on the touch panel display 13, the display control unit 37 may be omitted.

### §3 Operational Example

### Control of Report Mode

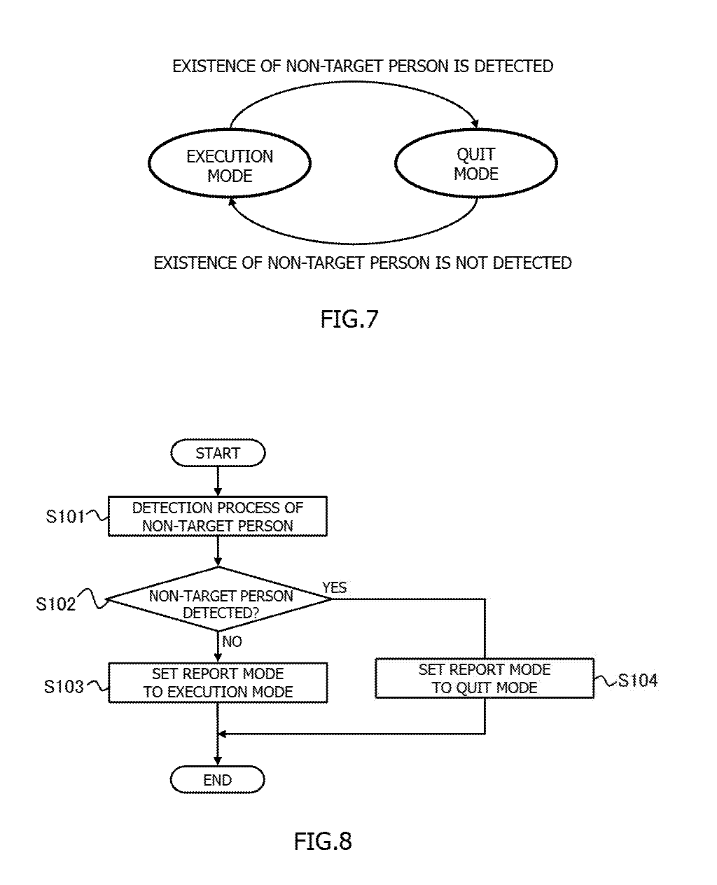

The discussion will start with describing a report mode for determining whether to execute the report to notify the user that the watching system detects the behavior by using FIGS. 7 and 8. However, a method of controlling whether or not the report is executed may not be limited to such a method but may be adequately selected corresponding to the embodiment.

FIG. 7 illustrates state transitions of the report mode according to the embodiment. As illustrated in FIG. 7, the report mode according to the embodiment is classified into two modes, i.e., an execution mode and a quit mode. Herein, the execution mode is a mode of executing the report to notify the user of the detection of the behavior. While on the other hand, the quit mode is a mode of quitting the execution of the report. Information representing the report mode is retained on, e.g., the RAM. The control unit 11 updates the information representing the report mode retained on the RAM, thereby switching over the report mode.

For instance, the control unit 11 sets the report mode to the execution mode just when starting watching. Upon detecting the existence of the non-target person, the control unit 11 switches over the report mode to the quit mode from the execution mode. Thereafter, the control unit 11 switches over the report mode to the execution mode from the quit mode when the existence of the non-target person is not detected. With this switchover, the control unit 11 controls the report mode of the watching system. Note that a processing procedure pertaining to the state transitions will be described by using FIG. 8.

FIG. 8 illustrates the processing procedure pertaining to the state transitions of the report mode according to the embodiment. The processing procedure to be hereinafter described is nothing but one example, and respective processes may be modified to the greatest possible degree. Steps of the processing procedure to be hereinafter described can be properly omitted, replaced and added corresponding to the embodiment.

(Step S101 & Step S102)

In step S101, the control unit 11 functions as the non-target person detection unit 35, and executes a process for detecting the existence of the non-target person in the range where the behavior of the watching target person can be watched. When detecting the existence of the non-target person in step S101, the control unit 11 diverts the processing to S104 from step S102. Whereas when not detecting the existence of the non-target person in step S101, the control unit 11 advances the processing to step S103 from step S102.

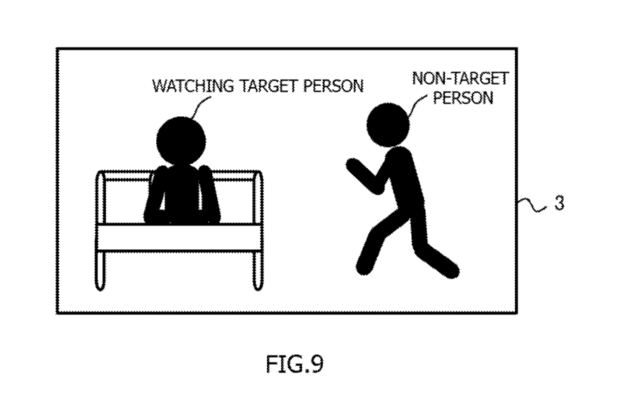

Note that the method of detecting the non-target person in the watching-enabled range may not be limited to a specific method but may be properly selected corresponding to the embodiment. In the embodiment, the control unit 11 detects the existence of the non-target person by analyzing the captured image 3. The method of detecting the existence of the non-target person by analyzing the captured image 3, will be described with reference to FIG. 9.

FIG. 9 schematically illustrates the captured image 3 containing the image of the non-target person. The captured image 3 contains the image of the non-target person, in which case the captured image 3 basically contains images of a plurality of persons. Accordingly, the control unit 11 determines whether the images of the plurality of persons are contained in the captured image 3, through an image analysis instanced by detecting patterns and graphic elements.

The control unit 11, when determining that the images of the plurality of persons are contained in the captured image 3, determines that the non-target person exists in the watching-enabled range. In this case, the control unit 11 therefore diverts the processing to step S104 from step S102. Whereas when determining that the images of the plurality of persons are not contained in the captured image 3, the control unit 11 determines that the non-target person does not exist in the watching-enabled range. In this case, the control unit 11 therefore advances the processing to step S103 from step S102.

According to this method, the image of the non-target person is contained in the captured image 3, whereby the control unit 11 detects the existence of the non-target person. The control unit 11 is thereby enabled to adequately grasp a period of having a possibility of causing the misreport of the watching system due to the non-target person's image being contained in the captured image 3. Hence, the control unit 11 detects the non-target person by analyzing the captured image 3 and is thereby enabled to suppress a below-mentioned report halt period down to the minimum.

Note that the image of the non-target person is contained in the captured image 3, in which case, as illustrated in FIG. 9, the non-target person exists in the vicinity of the watching target person. Consequently, in this case, the non-target person can watch at least the watching target person. In other words, the watching-enabled range of the watching target person covers the real-space range having the possibility of the non-target person's image being contained in the captured image 3.

Herein, the real-space range having the possibility of the non-target person's image being contained in the captured image 3 may set equivalent to the watching-enabled range itself of the watching target person, and may also set equivalent to part of the watching-enabled range of the watching target person. The watching-enabled range of the watching target person may be properly set. In the embodiment, the range of the non-target person's image being contained in the captured image 3 is set to the watching-enabled range of the watching target person.

(Step S103)

In step S103, the control unit 11 sets the report mode to the execution mode, and finishes the processes according to the present operational example. For instance, when the report mode has been set to the execution mode, the control unit 11 keeps the report mode being set to the execution mode. Whereas when the report mode has been set to the quit mode, the control unit 11 switches over the report mode to the execution mode from the quit mode.

(Step S104)

In step S104, the control unit 11 sets the report mode to the quit mode, and finishes the processes according to the present operational example. For example, when the report mode has been set to the quit mode, the control unit 11 keeps the report mode being set to the quit mode. Whereas when the report mode has been set to the execution mode, the control unit 11 switches over the report mode to the quit mode from the execution mode. The information processing apparatus 1 according to the embodiment thus controls the report mode of the watching system.

[Detection of Behavior of Watching Target Person]

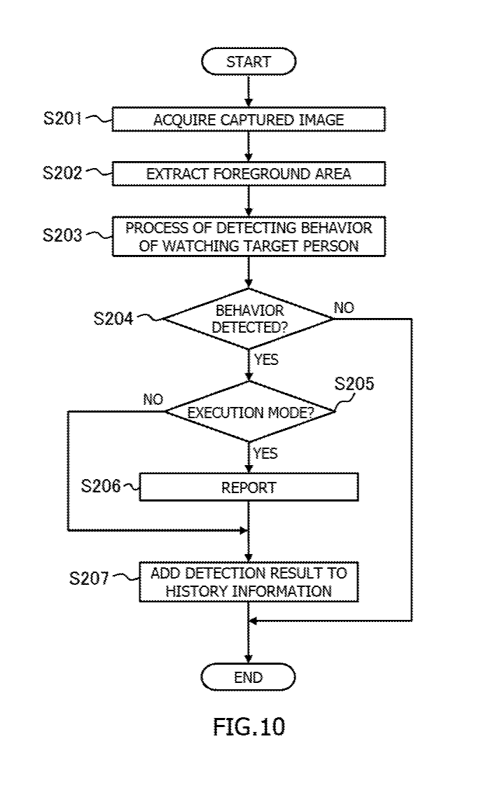

Next, a processing procedure of how the information processing apparatus 1 detects the behavior of the watching target person, will be described by using FIGS. 10 and 11. FIG. 10 illustrates the processing procedure of how the information processing apparatus 1 detects the behavior of the watching target person. FIG. 11 depicts a screen 50 displayed on the touch panel display 13 when executing the process pertaining to the behavior detection. It is to be noted that the process pertaining to the behavior detection will be described as a separate process from the process pertaining to the control of the report mode in the embodiment. However, the process pertaining to the detection of the behavior of the watching target person may also be executed in series of the process pertaining to the control of the report mode.

The control unit 11 according to the embodiment functions as the display control unit 37 when watching the watching target person in the processing procedure illustrated in FIG. 10, and displays the screen 50 depicted in FIG. 11 on the touch panel display 13. The screen 50 contains an area 51 for displaying the captured image 3 captured by the camera 2, a button 52 for accepting suspension of the watching process illustrated in FIG. 10, and a button 53 for accepting a variety of settings of the watching process.

The control unit 11 detects the behaviors related to the bed of the watching target person by executing the following processes in steps S201-S207 while displaying the screen 50 on the touch panel display 13. The user of the watching system watches the watching target person by making use of a detection result of the behavior in the watching system.

Note that a below-mentioned processing procedure pertaining to the behavior detection is nothing but one example, and the respective processes may be modified to the greatest possible degree. Steps of the below-mentioned processing procedure can be properly omitted, replaced and added corresponding to the embodiment. The screen displayed on the touch panel display 13 may not be limited to the screen 50 depicted in FIG. 7 but may be adequately set corresponding to the embodiment.

(Step S201)

In step S201, the control unit 11 functions as the image acquiring unit 31 and acquires the captured image 3 captured by the camera 2. In the embodiment, the camera 2 is equipped with the depth sensor 21. Consequently, the captured image 3 acquired in step S201 contains the depth information representing the depths of the respective pixels. The control unit 11 acquires, as the captured image 3 containing this depth information, the captured image 3 with the pixel gray values (pixel values) being determined corresponding to the depths of the respective pixels as illustrated in, e.g., FIGS. 5 and 11. In other words, the gray values of respective pixels of the captured image 3 illustrated in FIGS. 5 and 11 correspond to depths of object images contained in the respective pixels.

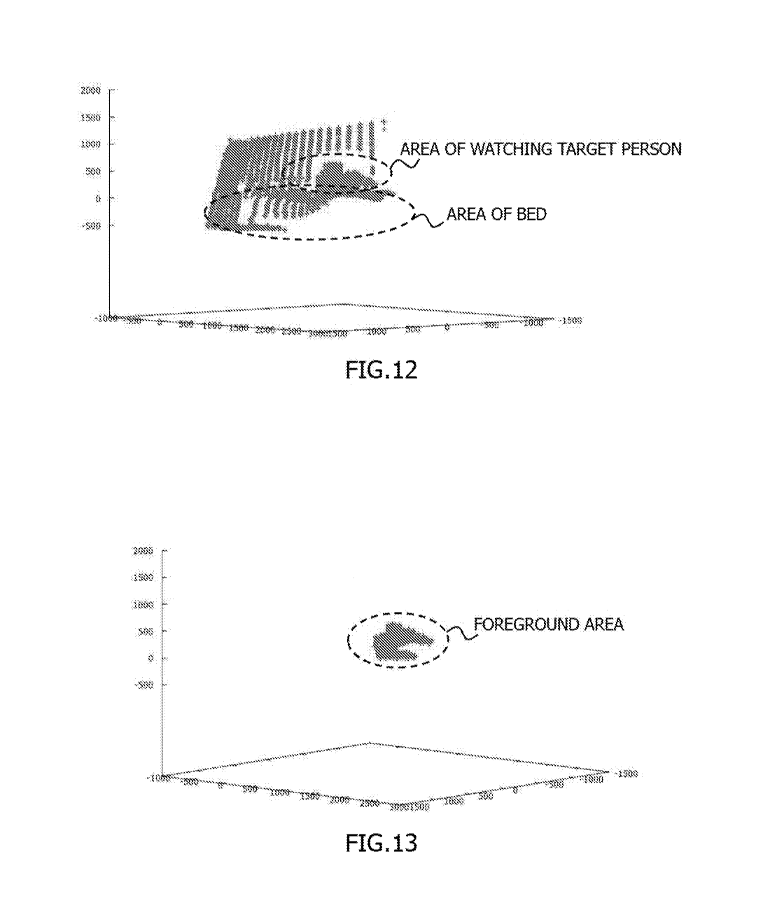

As described above, the control unit 11 can specify, based on the depth information, positions of the object images contained in the respective pixels in the real space. To be specific, the control unit 11 can specify the positions of the object images contained in the respective pixels in the three-dimensional space (real space) from the positions (two-dimensional information) and the depths of the respective pixels within the captured image 3. For example, states of the object images contained in the captured image 3 illustrated in FIG. 11 are depicted in FIG. 12 drawn next.

FIG. 12 depicts a three-dimensional distribution of the positions of the objects within the image capture range, which are specified based on the depth information contained in the captured image 3. The three-dimensional distribution depicted in FIG. 12 can be generated by plotting the respective pixels within the three-dimensional space with respect to the positions and the depths in the captured image 3. In other words, the control unit 11 can recognize the states of the object images contained in the captured image 3 in the real space as indicated by the three-dimensional distribution illustrated in FIG. 12.

It should be noted that the information processing apparatus 1 according to the embodiment is utilized for watching the inpatient in the medical treatment facility or the assisted-living resident in the nursing-care facility. Such being the case, the control unit 11 may acquire the captured image 3 by synchronizing with video signals of the camera 2 so that the behaviors of the inpatient or the assisted-living resident can be watched in real time. The control unit 11 may promptly execute processes in steps S202-S207, which will be described later on, for the acquired captured image 3. The information processing apparatus 1 attains real-time image processing by consecutively performing these operations without interruption, and is thereby enabled to watch the behaviors of the inpatient or the assisted-living resident.

(Step S202)

Referring back to FIG. 10, in step S202, the control unit 11 functions as the foreground extraction unit 32, and extracts the foreground area of the captured image 3 from a difference between a background image set as the background of the captured image 3 acquired in step S201 and the captured image 3. Herein, the background image is defined as data used for extracting the foreground area and is set including a depth of an object becoming the background. A method of generating the background image may be properly set corresponding to the embodiment. For example, the control unit 11 may generate the background image by calculating an average of several frames of the captured image, which are obtained when starting the watch of the watching target person. At this time, the average of the frames of the captured image is calculated including the depth information, thereby generating the background image inclusive of the depth information.

FIG. 13 illustrates the three-dimensional distribution of the foreground area extracted from the captured image 3 depicted in FIGS. 11 and 12. To be specific, FIG. 13 illustrates the three-dimensional distribution of the foreground area extracted when the watching target person gets up from on the bed. The foreground area extracted by making use of the background image described above appears in a position of having varied from the state indicated in the background image within the real space. Consequently, an area covering an image of a motion region of the watching target person is extracted as this foreground area, corresponding to the behavior taken by the watching target person. For instance, in FIG. 13, the watching target person takes such a motion that an upper part of the body rises (get-up state), and hence the area covering the image of the upper part of the body of the watching target person is extracted as the foreground area. The control unit 11 determines the motion of the watching target person by making use of this foreground area.

It should be noted that a method by which the control unit 11 extracts the foreground area may not be limited to the method described above in step S202, but the background and the foreground may be separated by using, e.g., a background differentiation method. The background differentiation method can be exemplified by a method of separating the background and the foreground from a difference between the aforementioned background image and the input image (the captured image 3); a method of separating the background and the foreground by use of three different images and a method of separating the background and the foreground by applying a statistic model. The method of extracting the foreground area may not be limited to a specific method but may be adequately selected corresponding to the embodiment.

(Step S203)

Referring back to FIG. 10, in step S203, the control unit 11 functions as the behavior detection unit 33 and determines, based on the depths of the respective pixels within the foreground area extracted in step S202, whether a positional relationship between the object image contained in the foreground area and the bed satisfies a predetermined condition. The control unit 11 detects the behavior conducted by the watching target person, based on a result of this determination.

Note that the method of detecting the behavior of the watching target person, the predetermined condition for detecting the individual behaviors and the detection target behaviors may not be particularly limited but may be properly selected corresponding to the embodiment. The following discussion will describe a method of detecting a get-up state, a leaving-bed state, a sitting-on-bed-edge state and an over-bed-fence state, based on a positional relationship between an upper surface of the bed and the foreground area, by way of one example of the method of detecting the behaviors of the watching target person.

Note that the upper surface of the bed is, as illustrated in FIG. 4, an upward surface of the bed in the vertical direction, e.g., an upper surface of a bed mattress. A range of this upper surface of the bed in the real space may be preset, may also be set by specifying a position of the bed from analyzing the captured image 3, and may further be set in such a manner that the user specifies this range within the captured image 3. Note that a reference object for detecting the behaviors of the watching target person may not be limited to the upper surface of the bed and may also be a virtual object without being confined to a physical object inherent in the bed.

To be specific, the control unit 11 according to the embodiment detects the behavior being conducted by the watching target person, based on the determination as to whether the positional relationship between the object image contained in the foreground area and the upper surface of the bed in the real space satisfies the predetermined condition. Therefore, the predetermined condition for detecting the behaviors of the watching target person corresponds to a condition for determining whether the object image contained in the foreground area is covered by a predetermined area (which will hereinafter be also termed a “detection area”) specified with the reference object being the upper surface of the bed. This being the case, for facilitating the explanation, the method of detecting the behaviors of the watching target person will be described based on a relationship between the detection area and the foreground area.

(1) Get-Up State

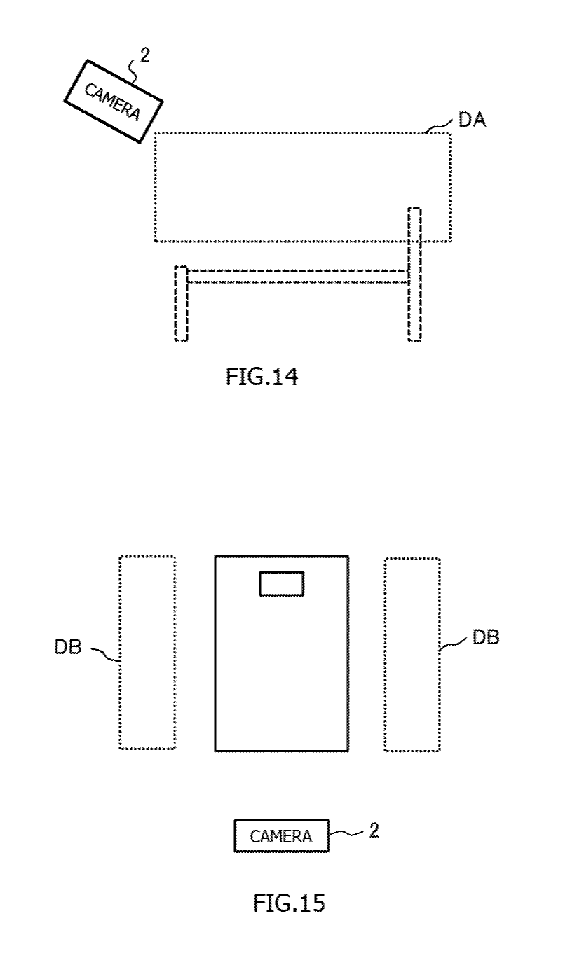

FIG. 14 schematically illustrates a detection area DA for detecting a get-up state. When the watching target person gets up from on the bed, an assumption is that the foreground area depicted in FIG. 13 appears above the upper surface of the bed. Accordingly, as illustrated in FIG. 14, the detection area DA for detecting the get-up state may be set in a position that is higher, e.g., by a predetermined upward distance in a heightwise direction than the upper surface of the bed. A set range of the detection area DA may not be limited to a specific range but may be properly determined corresponding to the embodiment. The control unit 11 detects the get-up state of the watching target person from on the bed when determining that the detection area DA covers the object image contained in the foreground area having a pixel count equal to or larger than, e.g., a threshold value.

(2) Leaving-Bed State

FIG. 15 schematically illustrates a detection area DB for detecting a leaving-bed state. When the watching target person leaves the bed, it is assumed that the foreground area appears in a position away from a side frame of the bed. Therefore, the detection area DB for detecting the leaving-bed state may be, e.g., as illustrated in FIG. 15, set in a position away in a widthwise direction of the bed from the upper surface of the bed. A set range of this detection area DB may be properly determined according to the embodiment similarly to the detection area DA. The control unit 11 detects the leaving-bed state of the watching target person when determining that the detection area DB covers the object image contained in the foreground area having the pixel count equal to or larger than, e.g., the threshold value.

(3) Sitting-on-Bed-Edge State

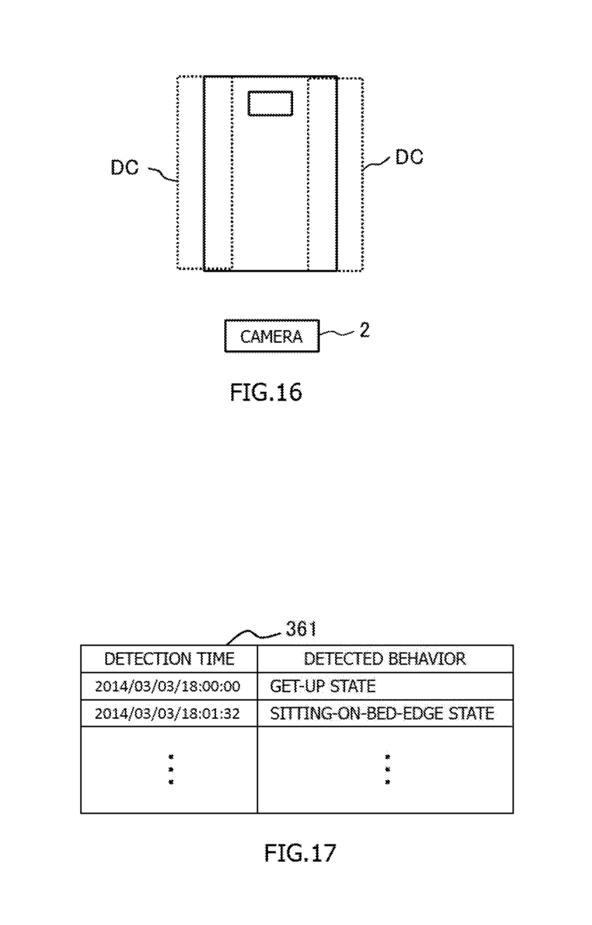

FIG. 16 schematically illustrates a detection area DC for detecting a sitting-on-bed-edge state. When the watching target person sits on the bed edge, it is assumed that the foreground area appears in the periphery of the side frame of the bed and from upward to downward of the bed. Accordingly, the detection area DC for detecting the sitting-on-bed-edge state may be, as depicted in FIG. 16, set in the periphery of the side frame of the bed and from upward to downward of the bed. The control unit 11 detects the sitting-on-bed-edge state of the watching target person when determining that the detection area DC covers the object image contained in the foreground area having the pixel count equal to or larger than, e.g., the threshold value.

(4) Over-Bed-Fence State

When the watching target person leans over a fence of the bed, in other words, when the watching target person moves over the fence of the bed, it is assumed that the foreground area appears in the periphery of the side frame of the bed and upward of the bed. Consequently, a detection area for detecting the over-bed-fence state may be set in the periphery of the side frame of the bed and upward of the bed. The control unit 11 detects the over-bed-fence state of the watching target person when determining that this detection area covers the object image contained in the foreground area having the pixel count equal to or larger than, e.g., the threshold value.

(5) Others

In step S203, the control unit 11 detects a variety of behaviors of the watching target person. Specifically, the control unit 11 can detect each behavior when determining that a determination condition for each behavior is satisfied. Whereas when determining that the determination condition for each behavior is not satisfied, the control unit 11 advances the processing to next step S204 without detecting the behavior of the watching target person.

It is to be noted that the method of detecting the behaviors of the watching target person may not be limited to the methods described above but may be properly selected corresponding to the embodiment. For example, the control unit 11 may calculate an average position of the foreground areas by taking an average of the positions and the depths of the respective pixels extracted as the foreground areas within the captured image 3. The control unit 11 may detect the behavior of the watching target person by determining whether the detection area set as the condition for detecting each behavior in the real space includes the average position of the foreground areas.

The control unit 11 may also specify a body region with its image being contained in the foreground area, based on a shape of the foreground area. The foreground area indicates a variation from the background area. Hence, the body region with its image being contained in the foreground area corresponds to the motion region of the watching target person. Based on this correspondence, the control unit 11 may detect the behavior of the watching target person on the basis of the positional relationship between the specified body region (motion region) and the upper surface of the bed. Similarly to this, the control unit 11 may also detect the behavior of the watching target person by determining whether the body region with its image being contained in the foreground area covered by the detection area of each behavior is a predetermined body region.

It should be noted that the bed according to the embodiment corresponds to “an object serving as a reference object for the behavior of the watching target person” according to the present invention. Such an object may not be limited to the bed but may be properly selected corresponding to the embodiment.

(Step S204)

In step S204, the control unit 11 determines whether the behavior of the watching target person can be detected in step S203. When the behavior of the watching target person is detected in step S203, the control unit 11 advances the processing to next step S205. Whereas when the behavior of the watching target person is not detected in step S203, the control unit 11 finishes the processes pertaining to the present operational example.

(Step S205)

In step S205, the control unit 11 functions are the report unit 34 and refers to information indicating the report mode. The control unit 11 determines whether the report mode of the watching system is the execution mode. The report mode of the watching system is the execution mode, in which case the control unit 11 advances the processing to next step S206 and executes the report to notify the user of the detection of the behavior. While on the other hand, the report mode of the watching system is not the execution mode, in other words, the report mode of the watching system is the quit mode, in which case the control unit 11 quits the process in step S206 and advances the processing to step S207.

Namely, the control unit 11 executes the report to notify the user of the detection of the behavior in next step S206, corresponding to the event that the behavior of the watching target person is detected in step S203, while the report mode of the watching system remains set to the execution mode. On the other hand, while the report mode of the watching system remains set to the quit mode, the execution of the report to notify the user of the detection of the behavior in step S206 is quitted even when detecting the behavior of the watching target person in step S203.

(Step S206)

In step S206, the control unit 11 functions as the report unit 34, and executes the report to notify the user of the detection of the behavior, corresponding to the detection of the behavior of the watching target person in step S203. A means by which the control unit 11 executes the report (which will hereinafter be also referred to as a “report means”) may be properly selected corresponding to the embodiment.

For example, the control unit 11 may execute the report to notify the user of the detection of the behavior of the watching target person by cooperating with the equipments installed in the facility instanced by the nurse call system 4 connected to the information processing apparatus 1. In this case, the control unit 11 may control the nurse call system 4 via the external interface 15. The control unit 11 may perform, e.g., the calling via the nurse call system 4 as the report to notify the user of the detection of the behavior. The user watching the behavior of the watching target person can be thereby adequately notified that the behavior of the watching target person has been detected.

Note that the control unit 11 causes at least any one of, e.g., the master unit 40 and the slave unit 41 to output a predetermined sound by way of one example of the calling via the nurse call system 4. This calling may be performed by both of the master unit 40 and the slave unit 41 and may also be performed by any one of these units. The calling method may be properly selected corresponding to the embodiment.

For instance, the control unit 11 may also execute the report to notify the user of the detection of the behavior of the watching target person by outputting the predetermined sound from the speaker 14 connected to the information processing apparatus 1. When the speaker 14 is disposed in the periphery of the bed, it is feasible to notify the person existing in the periphery of a watching place that the behavior of the watching target person has been detected by performing the report via the speaker 14. The person existing in the periphery of the watching place may also be encompassed by the watching target person himself or herself. The watching system is thereby enabled to notify the watching target person himself or herself of an event that the watching target behavior has been taken.

For example, the control unit 11 may display a screen for notifying the user of the detection of the behavior of the watching target person on the touch panel display 13. The control unit 11 may also give such notification by using, e.g., an e-mail. In this case, an e-mail address of a user terminal as a recipient of the notification may be previously registered in the storage unit 12, and the control unit 11 may execute the report to notify the user of the detection of the behavior of the watching target person by using the pre-registered e-mail address.

Note that the report to notify the user of the detection of the behavior of the watching target person may also be conducted as the notification to inform the watching target person of a symptom of encountering with an impending danger. In this case, the control unit 11 may not execute the report about the overall detected behaviors. For example, if the behavior detected in, e.g., step S203 is a behavior indicating the symptom of encountering with the impending danger, the control unit 11 may execute this report.

Herein, the behavior set as the behavior indicating the symptom of encountering with the impending danger may be properly selected corresponding to the embodiment. For instance, the sitting-on-bed-edge state may be set as the behavior indicating the symptom that the watching target person will encounter with the impending danger, i.e., as the behavior having a possibility that the watching target person will come down or fall down. In this case, the control unit 11, when detecting in step S203 that the watching target person is in the sitting-on-bed-edge state, determines that the behavior detected in step S203 is the behavior indicating the symptom that the watching target person will encounter with the impending danger.

When determining whether the behavior detected in step S203 is the behavior indicating the symptom that the watching target person will encounter with the impending danger, the control unit 11 may make use of transitions of the behaviors of the watching target person. For example, a possibility that the watching target person will come down or fall down is assumed higher in a transition to the sitting-on-bed-edge state from the get-up state than a transition to the sitting-on-bed-edge state from the leaving-bed state. Such being the case, the control unit 11 may determine, based on the transitions of the behaviors of the watching target person, whether the behavior detected in step S203 is the behavior indicating the symptom that the watching target person will encounter with the impending danger.

For instance, it is assumed that the control unit 11 detect the get-up state of the watching target person in step S203 during the periodic detection of the behaviors of the watching target person and thereafter detects the transition to the sitting-on-bed-edge state of the watching target person. Hereat, the control unit 11 may determine in step S206 that the behavior presumed in step S203 is the behavior indicating the symptom that the watching target person will encounter with the impending danger.

(Step S207)

In step S207, the control unit 11 functions as the history generation unit 36 and generates the history of the behaviors of the watching target person, which are detected in step S203. For example, the control unit 11 generates the history information of the behaviors, detected in step S203, of the watching target person, and causes the storage unit 12 to retain the generated history information. The history of the behaviors of the watching target person may be thus generated.

Herein, the history information generated in step S207 will be described by using FIG. 17. FIG. 17 illustrates the history information in a table format. A data format of the history information may not be, however, limited to the table formation illustrated in FIG. 17, and may be properly selected corresponding to the embodiment.

In the history information depicted in FIG. 17, a content of one record corresponds to a result of the detection conducted once. Each record of the history information contains a field to enter “detection time” and a field to enter the “detected behavior”. The detection time indicates time when the behavior of the watching target person is detected in step S203. The detected behavior indicates a type of the behavior detected in step S203.

In step S207, the control unit 11 at first reads the history information retained in the storage unit 12 into the RAM. The control unit 11 generates the record associated with the detection result in step S203, and adds the generated record to the history information being read into the RAM. Finally, the control unit 11 writes the history information receiving the addition of the detection result to the storage unit 12. The history information is thereby kept updating whenever the behavior of the watching target person is detected.

As discussed above, in the embodiment, it is possible to archive the history of the behaviors detected by the watching system. This history information enables the user to retrospectively check the past behaviors of the watching target person.

Note that the behavior detection process for the watching target person in step S203 is executed even when the report mode is the quit mode in the embodiment. A result of the behavior detection process is stored in step S207. The following is a reason why not to quit the process of detecting the behavior of the watching target person and the history generation process thereof even when in the quite mode as described above.

Namely, the non-target person entering the watching-enabled range does not necessarily pay attention to the watching target person. There is a possibility that the non-target person pays attention to something or somebody other than the watching target person. Taking such a possibility into consideration, it is therefore better not to quit the behavior detection process itself for the watching target person even when stopping executing the report to notify the user of the detection of the behavior. To support this policy, according to the embodiment, the control unit 11 detects the behavior of the watching target person irrespective of whether the existence of the non-target person is detected. The control unit 11 generates the history of the detected behaviors of the watching target person regardless of whether the existence of the non-target person is detected.

However, this detection result has a possibility of being erroneous due to the existence of the non-target person. This being the case, for enabling a determination as to whether the detection result is valid, the control unit 11 may store the captured image 3 acquired when detecting the behavior of the watching target person in the storage unit 12 by being associated with the record containing this detection result. The user can determine whether the detection result of the behavior of the watching target person is valid by referring to the captured image 3 stored together with the history.

Upon completing this history generation process, the control unit 11 finishes the processes pertaining to the present operational example. The information processing apparatus 1 may periodically repeat the processes given in the aforementioned operational example in the case of periodically detecting the behaviors of the watching target person. An interval of periodically repeating the process may be properly set. The information processing apparatus 1 may also execute the processes given in the aforementioned operational example in response to a request of the user. The information processing apparatus 1 may further suspend the processes pertaining to the detection of the behaviors of the watching target person, corresponding to an operation of the button 52 provided on the screen 50.

As discussed above, the information processing apparatus 1 according to the embodiment quits executing the report to notify the user of the detection of the behavior of the watching target person in a condition enabling the existence of the non-target person to be detected in the range where the behavior of the watching target person can be detected. More specifically, the information processing apparatus 1 quits executing the report to notify the user of the detection of the behavior when the existence of the non-target person is detectable within the captured image 3. Consequently, in such a scene that the captured image 3 contains the image of the non-target person other than the watching target person, the watching system does not execute the report. It is thereby feasible to prevent the misreport of the watching system due to the non-target person's image being contained in the captured image 3.

The information processing apparatus 1 according to the embodiment evaluates the positional relationship between the motion region of the watching target person and the bed in the real space by making use of the foreground area and the depths of the object, thereby detecting the behaviors of the watching target person. Namely, the information processing apparatus 1 according to the embodiment detects the behaviors of the watching target person by making use of the depth information. According to the embodiment, it is therefore possible to presume the behavior suited to the state of the watching target person in the real space.

The information processing apparatus 1 according to the embodiment specifies the position of the motion region of the watching target person by making use of the foreground area extracted in step S202. Herein, the process for extracting the foreground area is a mere process of calculating the difference between the captured image 3 and the background area. Hence, according to the embodiment, the control unit 11 (the information processing apparatus 1) can detect the behaviors of the watching target person without employing the high-level image processing. The processes pertaining to the detection of the behaviors of the watching target person can be thereby speeded up.

### §4 Modified Example

The in-depth description of the embodiment of the present invention has been made so far but is no more than the exemplification of the present invention in every point. The present invention can be, as a matter of course, improved and modified in the variety of forms without deviating from the scope of the present invention.

(1) Utilization of Planar Dimension

For example, the image of the object within the captured image 3 becomes smaller as the object gets remoter from the camera 2 but becomes larger as the object gets closer to the camera 2. The depth of the object image contained in the captured image 3 is acquired based on the surface of the object and a planar dimension of the surface of the object corresponding to the respective pixels in the captured image 3 is not, however, necessarily coincident among the respective pixels.

Such being the case, for eliminating influence caused by far-to-near positions of the object, the control unit 11 may calculate a planar dimension of a partial region, covered by the detection area, of the object image contained in the foreground area in the real space in step S203. Then, the control unit 11 may detect the behavior of the watching target person on the basis of the calculated planar dimension.

Note that the planar dimension of each pixel within the captured image 3 in the real space can be obtained based on the depth of each pixel as follows. The control unit 11 can respectively calculate a lateral length w and a longitudinal length h of an arbitrary point s (1 pixel) within the captured image 3 in the real space as illustrated in FIG. 11 on the basis of a relational expression of Mathematical Expression 1 and Mathematical Expression 2 that are given below. Note that Dₛ designates a depth at the point s. A symbol Vx represents an angle of view of the camera 2 in the crosswise direction. A symbol V_{y} stands for an angle of view of the camera 2 in the lengthwise direction. A symbol W indicates a pixel count of the captured image 3 in the crosswise direction. A symbol H designates a pixel count of the captured image 3 in the lengthwise direction. A coordinate of a central point (pixel) of the captured image 3 is to be (0, 0). The control unit 11 can acquire, e.g., these items of information by accessing the camera 2.

$$\begin{matrix}w = ( {D}_{s} \times tan \frac{{V}_{x}}{2} ) \text{/} \frac{W}{2} & ( Mathematical Expression 1 ) \\ h = ( {D}_{s} \times tan \frac{{V}_{y}}{2} ) \text{/} \frac{H}{2} & ( Mathematical Expression 2 )\end{matrix}$$

Accordingly, the control unit 11 can obtain the planar dimension of 1 pixel in the real space at the depth Dₛ by a square of the thus-calculated lateral length w, a square of the thus-calculated longitudinal length h, or a product of w and h. Then, in step S203, the control unit 11 calculates a total sum of the planar dimensions of the respective pixels of the object image contained in the detection area in the real space in the pixels within the foreground area. The control unit 11 may also detect the behavior of the watching target person on the bed by determining whether the calculated total sum of the planar dimensions is encompassed by a predetermined range. Detection accuracy of the behavior of the watching target person can be thereby enhanced, while eliminating the influence caused by the far-to-near positions of the object.

Note that these planar dimensions are to largely vary depending on a noise of the depth information, a motion of an object other than the watching target person, and other equivalent factors as the case may be. To cope with this variation, the control unit 11 may utilize an average of planar dimensions of several frames. Further, if a difference between a planar dimension of the relevant area in a processing target frame and an average of the planar dimensions of the relevant areas in the past several frames older than this processing target frame exceeds a predetermined range, the control unit 11 may exclude the relevant area from the processing target.

(2) Behavior Presumption Utilizing Planar Dimension and Dispersion

In the case of detecting the behavior of the watching target person by utilizing the planar dimension described above, a range of the planar dimension serving as a condition for detecting the behavior is set based on a predetermined region, presumed to be contained in the detection area, of the watching target person. The predetermined region is exemplified by a head, a shoulder and other equivalent regions of the watching target person. In other words, the range of the planar dimension serving as the condition for detecting the behavior is set based on the planar dimension of the predetermined region of the watching target person.

The control unit 11 is, however, unable to specify a shape of the object image contained in the foreground area from only the planar dimension of the object image contained in the foreground area in the real space. Consequently, the control unit 11 has a possibility of mistaking the body region, covered by the detection area, of the watching target person, resulting in erroneously detecting the behavior of the watching target person. Such being the case, the control unit 11 may prevent this mis-detection by utilizing a dispersion indicating a degree of spread in the real space.

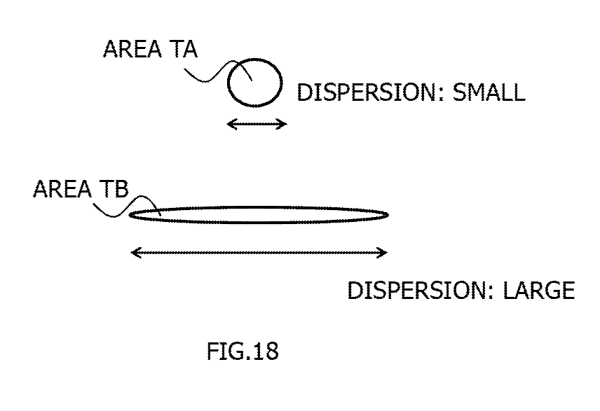

This dispersion will be described by using FIG. 18. FIG. 18 illustrates a relationship between the degree of spread of the area and the dispersion. Both of an area TA and an area TB illustrated in FIG. 18 are to have the same planar dimension. When trying to presume the behavior of the watching target person from only this planar dimension, the control unit 11 results in recognizing that the area TA is identical with the area TB, and therefore has the possibility of erroneously detecting the behavior of the watching target person.

However, as illustrated in FIG. 18, the area TA and the area TB are largely different in their spreads in the real space (the different degrees of spreads in the horizontal direction in FIG. 18). This being the case, in step S203, the control unit 11 may calculate the dispersion of each of the pixels contained in the detection area in the pixels contained in the foreground area. The control unit 11 may also detect the behavior of the watching target person on the basis of determining whether the calculated dispersion is encompassed by the predetermined range.

Note that a dispersion range serving as a condition for detecting the behavior is, similarly to the example of the planar dimension described above, set based on the predetermined region, presumed to be covered by the detection area, of the watching target person. For example, the predetermined region covered by the detection area is presumed to be the head, in which case a dispersion value serving as the condition for detecting the behavior is set within a range of substantially small values. On the other hand, the predetermined region covered by the detection area is presumed to be the shoulder, in which case the dispersion value serving as the condition for detecting the behavior is set within a range of substantially large values.

(3) Non-Utilization of Foreground Area

According to the embodiment, the control unit 11 (the information processing apparatus 1) detects the behavior of the watching target person by making use of the foreground area extracted in step S202. However, the method of detecting the behavior of the watching target person may not be limited to the method that utilizes the foreground area described above but may be properly selected corresponding to the embodiment.

Note that the control unit 11 may omit the process in step S202 in the case of not utilizing the foreground area for the process (step S203) of detecting the behavior of the watching target person. In this case, the control unit 11 functions as the behavior detection unit 33, and may detect the behavior pertaining to the bed of the watching target person by determining, based on the depth of each of the pixels within the captured image 3, whether the positional relationship between the watching target person and the bed in the real space satisfies the predetermined condition.

By way of this example, e.g., the control unit 11 may specify the area covering the image of the watching target person within the captured image 3 by an image analysis that involves detecting the pattern, the graphic element and other equivalent elements as the process in step S203. This area to be specified may contain an image of the whole body of the watching target person, and may also contain an image of one body region such as the head and the shoulder or images a plurality of body regions of the watching target person. The depths of the respective pixels contained in this specified area are utilized, whereby the position of the watching target person in the real space can be specified. Therefore, the control unit 11 may determine whether the positional relation in the real space between the watching target person and the bed satisfies the predetermined condition by utilizing the depths of the respective pixels contained in the specified area. The control unit 11 is thereby enabled to detect the behaviors pertaining to the bed of the watching target person.

(4) Non-Utilization of Depth Information

In the embodiment, the control unit 11 (the information processing apparatus 1) presumes the states of the watching target person in the real space on the basis of the depth information, thereby detecting the behaviors of the watching target person. The method of detecting the behaviors of the watching target person may not, however, be limited to the method that utilizes the depth information described above and may be properly selected corresponding to the embodiment.

In the case of not utilizing the depth information for the process (step S203) of detecting the behaviors of the watching target person, the camera 2 may not include the depth sensor 21. In this case, the control unit 11 functions as the behavior detection unit 33, and may detect the behavior of the watching target person by determining whether the positional relation between the watching target person and the bed, of which images are contained in the captured image 3, satisfies the predetermined condition. For example, the control unit 11 may specify the area covering the image of the watching target person by, similarly to the techniques described above, the image analysis that involves detecting the pattern, the graphic element and other equivalent elements. The control unit 11 may detect the behaviors pertaining to the bed of the watching target person on the basis of the positional relationship between the specified area covering the image of the watching target person and the specified area covering the image of the bed within the captured image 3. The control unit 11 may also detect the behavior of the watching target person by determining whether a position, in which the foreground area appears, satisfies the predetermined condition on the assumption that an object with its image being contained in the foreground area is to be the watching target person.

(5) Non-Target Person's Detection Utilizing Foreground Area

The non-target person entering the watching-enabled range is considered to be moving. Hence, the information processing apparatus 1 may detect the non-target person by utilizing the foreground area extracted in the process of step S202. In this case, the control unit 11 (the non-target person detection unit 35) may detect the existence of the non-target person by determining whether the foreground area encompasses an area covering the image of the non-target person through, e.g., the image analysis that involves detecting the pattern, the graphic element and other equivalent elements in step S101.

The control unit 11, when determining that the foreground area encompasses the area covering the image of the non-target person, diverts the processing to step S104, and may set the report mode to the quit mode. Whereas when determining that the foreground area does not encompass the area covering the image of the non-target person, the control unit 11 advances the processing to step S103, and may set the report mode to the execution mode.

Thus, the area for detecting the non-target person within the captured image 3 can be confined by utilizing the foreground area for detecting the non-target person. To be specific, the target area of the image analysis instanced by pattern matching is confined not within the overall area of the captured image 3 but within the foreground area. A load on the process in step S101 can be thereby reduced.

(6) Non-Target Person's Detection Utilizing Wireless Communication

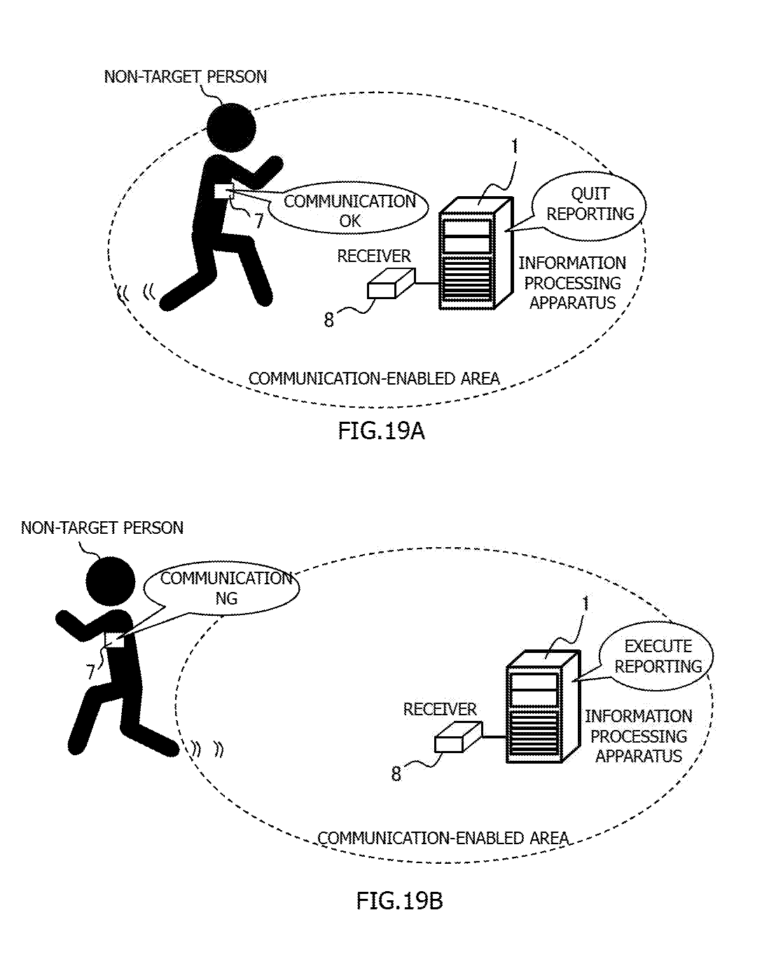

In the embodiment, the control unit 11 (the information processing apparatus 1) detects the existence of the non-target person other than the watching target person by analyzing the captured image 3 to be captured by the camera 2. The method of detecting whether the watching target person exists in the range where the watching target person can be watched, may not, however, be limited to the method based on the image analysis described above and may be properly selected corresponding to the embodiment. For example, the control unit 11 may execute a process of detecting the non-target person by utilizing wireless communications illustrated in FIGS. 19A and 19B in place of or together with the process in step S101.

FIGS. 19A and 19B illustrate scenes of detecting the non-target person by utilizing the wireless communications. The non-target person carries a wireless communication device 7 in the scenes illustrated in FIGS. 19A and 19B. The information processing apparatus 1 receives a connection of a receiver 8 for receiving information transmitted from this wireless communication device 7. The receiver 8 is disposed in a communication-enabled place with the wireless communication device 7 carried by the non-target person existing in the range where the watching target person can be watched. The place for disposing the receiver 8 may be properly selected corresponding to the embodiment, and the receiver 8 is disposed, e.g., under the bed. A communicable distance between the wireless communication device 7 and the receiver 8 is adequately adjusted so that a communication-enabled area corresponds to the range where the watching target person can be watched. The information processing apparatus 1 is thereby enabled to detect the existence of the non-target person in the range where the watching target person can be watched corresponding to an event that the receiver 8 receives the information transmitted from the wireless communication device 7.

FIG. 19A illustrates such a scene that the non-target person enters an area where the wireless communication device 7 and the receiver 8 can perform the communications with each other (“communication-enabled area” in FIG. 19A), and the wireless communication device 7 is thereby enabled to communicate with the receiver 8. In this scene, in step S101, the control unit 11 (the non-target person detection unit 35) can detect the existence of the non-target person, corresponding to the event that the receiver 8 receives the information for the wireless communication device 7. Consequently, the control unit 11 makes a determination of having detected the non-target person in step S102, and diverts the processing to step S104. Therefore, the control unit 11 sets the report mode of the watching system to the quit mode. With this quit mode being set, the information processing apparatus 1 quits executing the report to notify the user of the detection of the behavior of the watching target person.

On the other hand, FIG. 19B illustrates such a scene that the non-target person leaves the communication-enabled area, and the wireless communication device 7 is thereby disabled from performing the communications with the receiver 8. In this scene, in step S101, the control unit 11 (the non-target person detection unit 35) cannot detect the existence of the non-target person because of the receiver 8 being disabled from performing the communications with the wireless communication device 7. The control unit 11 therefore determines in step S102 that the existence of the non-target person is not detected, and advances the processing to step S103. Namely, the control unit 11 sets the report mode of the watching system to the execution mode. With this execution mode being set, the information processing apparatus 1 executes the report to notify the user of the detection of the behavior of the watching target person, corresponding to the detection of the behavior of the watching target person.

In this example, even when the captured image 3 contains none of the non-target person's image, the information processing apparatus 1 has a possibility of detecting the existence of the non-target person and quitting the execution of the report to notify the user of the detection of the behavior. Accordingly, the quitting of the report in such a scene does not contribute to reducing the misreport. However, as described above, in terms of the operation to watch the watching target person, there is a substantially low possibility of causing a hindrance against watching the watching even when quitting the report in such a scene. Therefore, the report may be quitted in the scene described above.

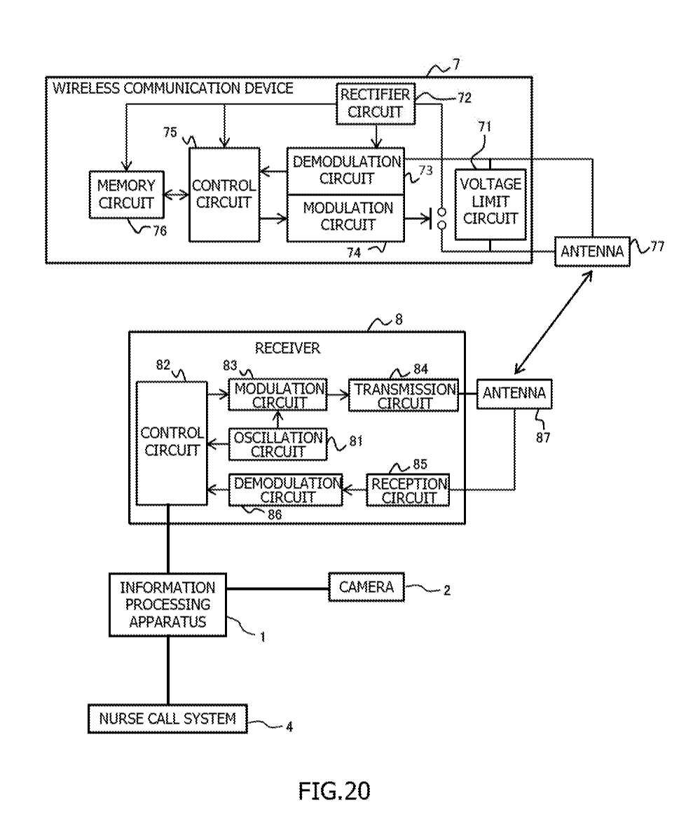

A hardware configuration will herein be described by using FIG. 20, of the watching system in the case of detecting the non-target person by utilizing the wireless communications. FIG. 20 illustrates the hardware configuration of the watching system in this case. Note that a hardware configuration of the information processing apparatus 1 is omitted in FIG. 20. The configuration depicted in FIG. 2 is applied to the hardware configuration of the information processing apparatus 1.

(Wireless Communication Device)

The wireless communication device 7 includes a voltage limit circuit 71, a rectifier circuit 72, a demodulation circuit 73, a modulation circuit 74, a control circuit 75, a memory circuit 76, and an antenna 77. Components of the specific hardware configuration of the wireless communication device 7 can be properly omitted, replaced and added corresponding to the embodiment.

The voltage limit circuit 71 is a circuit for protecting internal circuits from an excessive input to the antenna 77. The rectifier circuit 72 converts an alternate current inputted to the antenna 77 into a direct current, and supplies the electric power to the internal circuits. The demodulation circuit 73 demodulates an AC signal inputted to the antenna 77, and transmits the demodulated signal to the control circuit 75. On the other hand, the modulation circuit 74 modulates the signal transmitted from the control circuit 75, and transmits information to the receiver 8 via the antenna 77.

The control circuit 75, which is a circuit for controlling the operation of the wireless communication device 7, performs a variety of arithmetic processes corresponding to the inputted signals. The control circuit 75 may be built up by a minimum number of necessary logic circuits for the purpose of suppressing power consumption, and may also be built up by a large-scale circuit instanced by a CPU (Central Processing Unit).

The memory circuit 76 is a circuit for recording the information. A memory area of this memory circuit 76 may contain an area for storing a unique ID of a tag and accepting only reading. Note that the memory circuit 76 can involve using, e.g., an EPROM (Electrically Programmable Read Only Memory), an EEPROM (Electrically Erasable and Programmable Read Only Memory), an FeRAM (Ferro electric Random Access Memory) and an SRAM (Static Random Access Memory).

Identifying information for identifying the non-target person carrying the wireless communication device 7 may also be written to the memory circuit 76. In this case, the control circuit 75 transmits this identifying information to the receiver 8 by properly controlling the modulation circuit 74. Herein, the identifying information may contain any content on condition that the non-target person can be identified from this information. For example, the content of the identifying information may also be a code allocated to the non-target person.

Note that the wireless communication device 7 illustrated in FIG. 20 is classified as an RFID (Radio Frequency IDentification) passive tag. However, a device capable of transmitting the information through the wireless communications may be sufficient as the wireless communication device 7, and devices other than the RFID tags may also be employed. A wireless-communication-enabled user terminal instanced by a mobile phone and a smartphone may also be used as the wireless communication device 7. A not passive but active RFID tag may further be used as the wireless communication device 7.

Telecommunications standards for the wireless communication device 7 may be properly selected corresponding to the embodiment. The telecommunications standards instanced by Wi-Fi (registered trademark), Bluetooth (registered trademark), ZigBee (registered trademark) and UWB (Ultra Wide Band) may be utilized as the telecommunications standard for the wireless communication device 7. It is, however, preferable to select the telecommunications standards not affecting medical devices instanced by a pacemaker in the case of utilizing the wireless communication device 7 in a medical setting as in the embodiment.

A mode of how the non-target person carries the wireless communication device 7 may also be properly determined corresponding to the embodiment. As illustrated in FIGS. 19A and 19B, the wireless communication device 7 (RFID tag) may be attached to a nameplate fitted in the vicinity of a non-target person's breast. Herein, basically the non-target person always carries the wireless communication device 7. Therefore, the wireless communication device 7 is preferably small in size and light in weight like the RF tag. These features can make it easy for the non-target person to carry the wireless communication device 7 and make it difficult for the non-target person to be aware of carrying the wireless communication device 7.

(Receiver)

The receiver 8 includes an oscillation circuit 81, a control circuit 82, a modulation circuit 83, a transmission circuit 84, a reception circuit 85, a demodulation circuit 86 and an antenna 87. Components of the specific hardware configuration of the receiver 8 can be properly omitted, replaced and added corresponding to the embodiment.

The oscillation circuit 81 generates carrier waves used for the communications with the wireless communication device 7. The control circuit 82 is connected to the information processing apparatus 1 via the external interface 15. The control circuit 82 controls the communications with the information processing apparatus 1, controls the communications with the wireless communication device 7, controls the operation of the receiver 8, and controls other equivalent elements.

The modulation circuit 83 modulates signals from the control circuit 82 by superposing these signals on the carrier waves generated by the oscillation circuit 81, and transmits the modulated signals to the transmission circuit 84. The transmission circuit 84 transmits the signals superposed on the carrier waves, which are transmitted from the modulation circuit 83, to the wireless communication device 7 via the antenna 87. On the other hand, the reception circuit 85 receives the carrier waves transmitted from the wireless communication device 7 and coming in via the antenna 87. The demodulation circuit 86 demodulates the signals superposed on the carrier waves received by the reception circuit 85, and transmits the demodulated signals to the control circuit 82.

Note that the receiver 8 illustrated in FIG. 20 is a reader/writer of the RFID. The receiver 8 may also, however, be a device other than the reader/writer if capable of receiving the information that is wirelessly transmitted from the wireless communication device 7.

(Operation and Effect)

The information processing apparatus 1 can detect the existence of the non-target person on the basis of whether the wireless communications are performed or not by utilizing the wireless communication device 7 and the receiver 8. In other words, the condition for detecting the existence of the non-target person is a mere piece of information about whether the wireless communications are performed or not, and hence the non-target person can be detected not depending on sophisticated information processing such as the image recognition.

The non-target person's detection based on the image analysis described above cannot be attained unless the captured image 3 contains the image of the non-target person. Consequently, there exists a possibility of causing a time lag till the non-target person is detected since the image of the non-target person gets contained in the captured image 3. Then, it follows that the information processing apparatus 1 executes, just when the non-target person behaves, detecting this behavior during this time lag, and such a possibility is caused that the information processing apparatus 1 executes a misreport of having detected the behavior of the watching target person.

By contrast, the method utilizing the wireless communications is capable of detecting the non-target person before the image of the non-target person gets contained in the captured image 3. Therefore, this method can prevent occurrence of the time lag till detecting the non-target person since the non-target person has got contained in the captured image 3. It is thereby feasible to prevent the misreport of the watching system having the possibility of causing the time lag.

(7) Identification of Non-Target Person

Note that the information processing apparatus 1 may set the report mode of the watching system to the quit mode in only such a case that the non-target person is identified based on the information transmitted from the wireless communication device 7 and the detected non-target person is a specified person. To be specific, in step S101, the control unit 11 (the non-target person detection unit 35) refers to the information transmitted from the wireless communication device 7 and may thus determine whether the non-target person entering the communication-enabled area is a predetermined person. The information used for this determination may be any types of information if usable for identifying the non-target person, and may be exemplified by the identifying information for identifying the non-target person and the ID unique to the tag.

The control unit 11 may divert the processing to step S104 when determining that the non-target person entering the communication-enabled area is the predetermined person. In other words, the control unit 11 may set the report mode of the watching system to the quit mode. While on the other hand, the control unit 11 may advance the processing to step S103 when the non-target person entering the communication-enabled area is not the predetermined person. In other words, the control unit 11 may set the report mode of the watching system to the execution mode. The information processing apparatus 1 is thereby enabled to quit executing the report to notify the user of the detection of the behavior only when the detected non-target person is the specified person.

According to the configuration described above, the execution of the report of the watching system is quitted only when the non-target person to be detected is the specified person. The person targeted for quitting the execution of the report of the watching system can be thus controlled. This control can prevent the execution of the report of the watching system from being quitted when the non-target person not paying attention to the behaviors of the watching target person enters the communication-enabled area.

(8) Selection of Report Means to Quit Executing Report

According to the embodiment discussed above, in such a scene that the watcher instanced by the nurse does not exist in the range where the watching target person can be watched, the nurse call system 4 is utilized as a report means for calling the watcher when the watching system detects the behavior of the watching target person. This calling is therefore unnecessary in such a scene that the watcher exists in the range where the watching target person can be watched. Hence, according to the embodiment, the information processing apparatus 1 quits executing the report of the watching system when the non-target person is detected in the range where the watching target person can be watched.

However, the non-target person existing in the watching-enabled range does not necessarily pay attention to the watching target person. For directing the attention of the non-target person to the watching target person, the information processing apparatus 1 may therefore start using another report means while quitting the execution of the report by the report means employed when the non-target person does not exist.

For example, the control unit 11 may perform calling via the slave unit 41 while quitting the calling via the master unit 40 of the nurse call system 4 in the quite mode. The control unit 11 may further display the screen for attracting the attention of the non-target person on the touch panel display 13 in the quit mode. The screen display for attracting the attention of the non-target person may include the screen display entailing a visual effect instanced by causing the screen to flicker. The control unit 11 may further output a sound for attracting the attention of the non-target person from the speaker 14 in the quit mode. The sound for attracting the attention of the non-target person may be a voice that utters a nomenclature of the detected behavior, and may also be an alarm sound like beeping. The attention of the non-target person existing in the watching-enabled range can be directed to the watching target person.

### REFERENCE SIGNS LIST

1 Information processing apparatus
2 Camera
3 Captured image
4 Nurse call system
5 Program
6 Storage medium
8 Depth sensor
9 Acceleration sensor
11 Control unit
12 Storage unit
13 Touch panel display
14 Speaker
15 External interface
16 Communication interface
17 Drive
31 Image acquiring unit
32 Foreground extraction unit
33 Behavior detection unit
34 Report unit
35 Non-target person detection unit
36 History generation unit
37 Display control unit

1. An information processing apparatus comprising:
an image acquiring unit configured to acquire a captured image captured by an imaging apparatus installed for watching a watching target person;
a behavior detection unit configured to detect a behavior of the watching target person by analyzing the acquired captured image;
a non-target person detection unit configured to detect an existence of a non-target person other than the watching target person in a range where the behaviors of the watching target person can be watched; and
a report unit configured to execute reporting to notify that the behavior of the watching target person is detected upon detecting the behavior of the watching target person while not detecting an existence of the non-target person, and to quit reporting to notify that the behavior of the watching target person is detected while detecting the existence of the non-target person.

2. The information processing apparatus according to claim 1, further comprising a history generation unit configured to generate a history of the behaviors of the watching target person, the behaviors being detected by the behavior detection unit.

3. The information processing apparatus according to claim 2, wherein the behavior detection unit detects the behaviors of the watching target person irrespective of whether the existence of the non-target person is detected or not, and
the history generation unit generates the history of the behaviors of the watching target person irrespective of whether the existence of the non-target person is detected or not, the behaviors being detected by the behavior detection unit.

4. The information processing apparatus according to claim 1, wherein the non-target person detection unit detects the existence of the non-target person by analyzing the acquired captured image.

5. The information processing apparatus according to claim 1, wherein the imaging apparatus captures an image of an object serving as a reference for the behaviors of the watching target person together with the image of the watching target person,
the image acquiring unit acquires the captured image containing depth information indicating a depth of each of pixels within the captured image, and the behavior detection unit detects the watching target person's behavior pertaining to the object by determining whether a positional relation in a real space between the watching target person and the object satisfies a predetermined condition or not, based on the depth of each of the pixels within the captured image, the depth being indicated by the depth information.

6. The information processing apparatus according to claim 5, further comprising a foreground extraction unit configured to extract a foreground area of the captured image from a difference between the captured image and a background image set as a background of the captured image,
the behavior detection unit detecting the watching target person's behavior pertaining to the object by determining whether the positional relation in the real space between the watching target person and the object satisfies the predetermined condition or not while utilizing, as a position of the watching target person, a position of the object in the real space with its image being contained in the foreground area, the latter position being specified based on the depth of each of the pixels in the foreground area.

7. The information processing apparatus according to claim 1, wherein the information processing apparatus is connected to a receiver to receive information transmitted from a wireless communication device carried by the non-target person, the receiver is disposed in a communication-enabled place with the wireless communication device carried by the non-target person existing in the range where the watching target person can be watched, and the non-target person detection unit detects the existence of the non-target person upon an event that the receiver receives the information transmitted from the wireless communication device.

8. The information processing apparatus according to claim 7, wherein the non-target person detection unit determines whether the non-target person is a predetermined person or not by referring to the information transmitted from the wireless communication device, and the report unit quits the report to notify that the behavior of the watching target person is detected when the non-target person is the predetermined person while detecting the existence of the non-target person.

9. The information processing apparatus according to claim 1, wherein the information processing apparatus is connected to a nurse call system for calling a person who watches the watching target person, and the report unit performs calling via the nurse call system as the report to notify that the behavior of the watching target person is detected.

10. An information processing method in which a computer executes: a step of acquiring a captured image captured by an imaging apparatus installed for watching a watching target person;
a step of detecting a behavior of the watching target person by analyzing the acquired captured image; a step of detecting an existence of a non-target person other than the watching target person in a range where the behaviors of the watching target person can be watched; a step of executing a report to notify that the behavior of the watching target person is detected upon detecting the behavior of the watching target person while not detecting an existence of the non-target person; and a step of quitting the report to notify that the behavior of the watching target person is detected while detecting the existence of the non-target person.

11. A non-transitory recording medium recording a program to cause a computer to execute: a step of acquiring a captured image captured by an imaging apparatus installed for watching a watching target person; a step of detecting a behavior of the watching target person by analyzing the acquired captured image;
a step of detecting an existence of a non-target person other than the watching target person in a range where the behaviors of the watching target person can be watched; a step of executing a report to notify that the behavior of the watching target person is detected upon detecting the behavior of the watching target person while not detecting an existence of the non-target person; and
a step of quitting the report to notify that the behavior of the watching target person is detected while detecting the existence of the non-target person.
